(19) United States
(12) Patent Application Publication
(10) Pub. No.: US 2018/0105596 A1
(43) Pub. Date: Apr. 19, 2018

(54) ANTI-TYRO3 ANTIBODIES AND USES THEREOF

(71) Applicants: ELSALYS BIOTECH, LYON (FR); CENTRE NATIONAL DE LA RECHERCHE SCIENTIFIQUE, PARIS (FR); INSTITUT CURIE, PARIS CEDEX 05 (FR)

(72) Inventors: VANESSA DUONG, OSTWALD (FR); THIERRY MENGUY, STRASBOURG (FR); HELENE HAEGEL, ILLKIRCH GRAFFENSTADEN (FR); ISABELLE BERNARD-PIERROT, NOGENT-SUR-MARNE (FR); FLORENT DUFOUR, MASSY (FR); FRANCOIS RADVANYI, FONTENAY-AUX-ROSES (FR)

(21) Appl. No.: 15/565,950

(22) PCT Filed: Apr. 15, 2016
(86) PCT No.: PCT/EP2016/058457

§ 371 Date: Oct. 12, 2017

(30) Foreign Application Priority Data
Apr. 17, 2015 (EP) 15305581.9

(51) Int. Cl. C07K 16/28; A61P 35/00; A61P 31/00

(52) U.S. Cl. CPC C07K 16/2863; A61P 35/00; A61P 31/00; C07K 2319/30; C07K 2317/24; C07K 2317/33; C07K 2317/622; C07K 2317/21

(57) ABSTRACT

The present invention relates to antibodies, in particular human or humanized antibodies, that bind to the extracellular domain of human Tyro3 receptor, in particular to the immunoglobulin-like domain Ig-1, and reduce or inhibit the binding of human Gas6 to said receptor. The invention also relates to the uses of these antibodies in the diagnosis, prevention or treatment of hyperproliferative or infectious diseases.

### FIELD OF THE INVENTION

The present invention relates to the field of medicine, in particular to antibodies which bind to the extracellular domain of the Tyro3 receptor tyrosine kinase. These antibodies exhibit advantageous characteristics, in particular for therapeutic and diagnostic purposes.

### BACKGROUND OF THE INVENTION

Tyro3, also known as Byk, Dtk, Rse, Rek, Sky or Tif, is a member of the Tyro3/Axl/Mer (TAM) family of receptor tyrosine kinases.

Tyrosine kinase receptors are composed of an extracellular domain, which is able to bind a specific ligand, a transmembrane domain, and an intracellular catalytic domain, which is able to bind and phosphorylate selected intracellular substrates. Binding of a ligand to the extracellular region causes a series of structural rearrangements in the tyrosine kinase receptor that lead to its enzymatic activation triggering a cascade of events through phosphorylation of intracellular proteins that ultimately transduces the extracellular signal to the nucleus, causing changes in gene expression.

The TAM receptors contain an N-terminal extracellular domain comprising two immunoglobulin (Ig)-related domains and two fibronectin type III (FNIII)-related domains followed by a single transmembrane helix and a cytoplasmic tyrosine kinase domain. Upon ligand binding, the receptors dimerize and the tyrosine kinases become activated.

These receptors can be activated by the homologous ligands Gas6 (growth arrest specific gene-6) and/or protein S which exhibit about 43% amino acid sequence identity and share the same complex multi-domain structure with the exception of thrombin cleavage sites which are present in Protein S but not Gas6.

Recent studies have revealed that the TAM receptors play key roles in immunity, hemostasis and inflammation (Zheng et al. J. Immunol 2015, 194; Rothlin et al. Cell. 2007, 131, 1124-1136). These receptors, and in particular Tyro3, were also shown to be associated with tumorigenesis and tumor cell survival (Linger et al. Adv Cancer Res, 2008; 100:35-83; Van der Meer et al., Blood. 2014; 213(16):2460-2469). Based on these findings, Tyro3 was identified as a target for the development of therapeutic agent for Tyro3 over-expressing cancers such as bladder cancer, melanoma or breast cancer (WO 2010/031828; Ekyalongo et al. Anticancer Res. 2014 July; 34(7):3337-45.) or to enhance the immune response (Lemke and Rothlin, Nat Rev Immunol. 2008; 8(5):327-336).

TAM receptor inhibitors include those molecules that reduce receptor activity such as those that specifically bind Gas6 or Protein S or extracellular domain and prevent the interaction between the ligand and the receptor, molecules that decrease Gas6 or protein S concentration, molecules that inhibit constitutive Tyro 3 activation and molecules that bind to the intracellular domain and prevent signalling of the receptor. Selective inhibitors may be either in the form of small molecules, soluble receptors lacking the tyrosine kinase domain, or antibodies.

The Tyro3 receptor is the least studied of the TAM receptors and, to date, there is no effective approaches for the therapeutic selective inhibition of this receptor. Some murine antibodies against Tyro3 are commercially available or have been described by Demarest et al (Biochemistry 2013, 52, 3102-3118) to study the activity of this receptor in melanoma. However, the use of murine monoclonal antibodies in the treatment of a human patient may result in a host immune response against the antibodies, thus compromising the efficacy of the treatment.

Accordingly, there is a strong need for human or humanized antibodies that effectively and specifically bind to Tyro3 and modulate its activity.

### SUMMARY OF THE INVENTION

The present invention provides anti-Tyro3 antibodies and methods of using the same.

In a first aspect, the present invention relates to an isolated human or humanized antibody that binds to the immunoglobulin-like domain Ig-1 of human Tyro3 receptor, does not bind to human Axl and Mer receptors, and reduces or inhibits the binding of human Gas6 to human Tyro3 receptor.

In particular, the antibody of the invention may comprise a CDR-L1 consisting, or consisting essentially, of the CDR-L1 of the light chain variable region of SEQ ID NO: 10 and/or comprises a CDR-H1 consisting, or consisting essentially, of the CDR-H1 of the heavy chain variable region of SEQ ID NO: 11.

The antibody may further bind to the immunoglobulin-like domain Ig-2 of human Tyro3. In particular, the antibody binding to the immunoglobulin-like domains Ig-1 and Ig-2 of human Tyro3 may comprise

a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 88, 102, 116, 130, 144 or 158, and

a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 89, 103, 117, 131, 145 or 159.

Alternatively, the antibody may bind to the immunoglobulin-like domain Ig-1 of humanTyro3 but not to the immunoglobulin-like domain Ig-2. In particular, this antibody may comprise

a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 10, 32, 46, 60 or 74, and

a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 11, 33, 47, 61 or 75.

In particular, the antibody of the invention may comprise

a light chain comprising CDR-L1, CDR-L2 and CDR-L3 consisting, or consisting essentiaéully, of CDR-L1, CDR-L2 and CDR-L3 of the light chain variable region of SEQ ID NO: 10, and a heavy chain comprising CDR-H1, CDR-H2 and CDR-H3 consisting, or consisting essentially, of CDR-H1, CDR-H2 and CDR-H3 of the heavy chain variable region of SEQ ID NO: 11, or

a light chain comprising CDR-L1, CDR-L2 and CDR-L3 consisting, or consisting essentially, of CDR-L1, CDR-L2 and CDR-L3 of the light chain variable region of SEQ ID NO: 32, and a heavy chain comprising CDR-H1, CDR-H2 and CDR-H3 consisting, or consisting essentially, of CDR-H1, CDR-H2 and CDR-H3 of the heavy chain variable region of SEQ ID NO: 33, or

a light chain comprising CDR-L1, CDR-L2 and CDR-L3 consisting, or consisting essentially, of CDR-L1, CDR-L2 and CDR-L3 of the light chain variable region of SEQ ID NO: 46, and a heavy chain comprising CDR-H1, CDR-H2 and CDR-H3 consisting, or consisting essentially, of CDR-H1, CDR-H2 and CDR-H3 of the heavy chain variable region of SEQ ID NO: 47, or

a light chain comprising CDR-L1, CDR-L2 and CDR-L3 consisting, or consisting essentially, of CDR-L1, CDR-L2 and CDR-L3 of the light chain variable region of SEQ ID NO: 60, and a heavy chain comprising CDR-H1, CDR-H2 and CDR-H3 consisting, or consisting essentially, of CDR-H1, CDR-H2 and CDR-H3 of the heavy chain variable region of SEQ ID NO: 61, or

a light chain comprising CDR-L1, CDR-L2 and CDR-L3 consisting, or consisting essentially, of CDR-L1, CDR-L2 and CDR-L3 of the light chain variable region of SEQ ID NO: 74, and a heavy chain comprising CDR-H1, CDR-H2 and CDR-H3 consisting, or consisting essentially, of CDR-H1, CDR-H2 and CDR-H3 of the heavy chain variable region of SEQ ID NO: 75, or

a light chain comprising CDR-L1, CDR-L2 and CDR-L3 consisting, or consisting essentially, of CDR-L1, CDR-L2 and CDR-L3 of the light chain variable region of SEQ ID NO: 88, and a heavy chain comprising CDR-H1, CDR-H2 and CDR-H3 consisting, or consisting essentially, of CDR-H1, CDR-H2 and CDR-H3 of the heavy chain variable region of SEQ ID NO: 89, or

a light chain comprising CDR-L1, CDR-L2 and CDR-L3 consisting, or consisting essentially, of CDR-L1, CDR-L2 and CDR-L3 of the light chain variable region of SEQ ID NO: 102, and a heavy chain comprising CDR-H1, CDR-H2 and CDR-H3 consisting, or consisting essentially, of CDR-H1, CDR-H2 and CDR-H3 of the heavy chain variable region of SEQ ID NO: 103, or

a light chain comprising CDR-L1, CDR-L2 and CDR-L3 consisting, or consisting essentially, of CDR-L1, CDR-L2 and CDR-L3 of the light chain variable region of SEQ ID NO: 116, and a heavy chain comprising CDR-H1, CDR-H2 and CDR-H3 consisting, or consisting essentially, of CDR-H1, CDR-H2 and CDR-H3 of the heavy chain variable region of SEQ ID NO: 117, or

a light chain comprising CDR-L1, CDR-L2 and CDR-L3 consisting, or consisting essentially, of CDR-L1, CDR-L2 and CDR-L3 of the light chain variable region of SEQ ID NO: 130, and a heavy chain comprising CDR-H1, CDR-H2 and CDR-H3 consisting, or consisting essentially, of CDR-H1, CDR-H2 and CDR-H3 of the heavy chain variable region of SEQ ID NO: 131, or

a light chain comprising CDR-L1, CDR-L2 and CDR-L3 consisting, or consisting essentially, of CDR-L1, CDR-L2 and CDR-L3 of the light chain variable region of SEQ ID NO: 144, and a heavy chain comprising CDR-H1, CDR-H2 and CDR-H3 consisting, or consisting essentially, of CDR-H1, CDR-H2 and CDR-H3 of the heavy chain variable region of SEQ ID NO: 145, or

a light chain comprising CDR-L1, CDR-L2 and CDR-L3 consisting, or consisting essentially, of CDR-L1, CDR-L2 and CDR-L3 of the light chain variable region of SEQ ID NO: 158, and a heavy chain comprising CDR-H1, CDR-H2 and CDR-H3 consisting, or consisting essentially, of CDR-H1, CDR-H2 and CDR-H3 of the heavy chain variable region of SEQ ID NO: 159.

The antibody may be an inhibitor of the Tyro3 receptor, preferably an inhibitor reducing or blocking the binding of a Tyro3 ligand with the extracellular domain of Tyro3, and/or reducing or blocking Tyro3 mediated signal transduction and/or reducing or blocking Tyro3 phosphorylation and/or reducing or blocking the Tyro3 dimerization.

The antibody may be an IgG1, IgG2, IgG3 or IgG4 antibody, preferably an IgG1 antibody.

In another aspect, the present invention relates to an isolated nucleic acid molecule selected from the group consisting of

(a) a nucleic acid sequence encoding an antibody of the invention, and

(b) a nucleic acid complementary to any of the sequences in (a).

The present invention also relates to a vector comprising a nucleic acid of the invention.

The present invention further relates to a host cell comprising a nucleic acid or a vector of the invention.

In a further aspect, the present invention relates to a method for producing an antibody of the invention, comprising culturing a host cell of the invention under conditions suitable for expression of the antibody, and optionally recovering said antibody.

In another aspect, the present invention relates to a pharmaceutical composition comprising an antibody, nucleic acid, vector or host cell of the invention, and a pharmaceutically acceptable carrier or excipient.

In a last aspect, the present invention also relates to an antibody or a pharmaceutical composition of the invention, for use in the diagnosis, prevention or treatment of a hyperproliferative disease or an infectious disease.

### BRIEF DESCRIPTION OF THE DRAWINGS

FIG. 1: scheme of the different fragments of human Tyro3 extracellular domain (ECD) that were produced.

FIG. 2: Double sandwich ELISA design.

FIG. 3: ELISA Binding assay of scFvs/IgG against hTyro 3 ECD. FIG. 3A: Results for ScFvs. FIG. 3B: results for IgGs.

FIG. 4: The purified extracellular domains of hTyro 3, hAxl or hMer were coated on microtiterplate and incubated in the presence of anti-Tyro 3 scFvs (1 μg/mL). FIG. 4A. 2410, 2411, 2412, 2413, 2414, 2415 scFvs. FIG. 4B. 2717 and 2718 scFvs. FIG. 4C. 2709, 2710, 2711 and 2713 scFvs. Bound fragments were revealed with an anti-Myc-HRP antibody.

FIG. 5: GAS6 competition assay with anti-Tyro3 scFvs. FIG. 5A. Binding of scFvs and MAB859 to full-length Tyro3. Full-length Tyro3 was coated onto an ELISA microtiter plate. scFvs were used at 10 μg/mL and MAB859 was used at 3 concentrations: 1 μg/mL (MAB859-1), 5 μg/mL (MAB859-5) and 10 μg/mL (MAB859-10). scFv binding to full-length Tyro3 was detected with an anti-HIS-HRP antibody and MAB859 with an anti-mouse-HRP antibody. The negative controls with the detection antibodies alone were shown (anti-HIS or anti-mouse). FIG. 5B. Effect of scFvs on Tyro3/GAS6 binding. Biotinylated GAS6 (2.5 μg/mL) alone or scFv/GAS6 mixtures were applied for 1 h at room temperature onto full-length Tyro3 coated plates. Binding of GAS6 to Tyro3 was detected with streptavidin-HRP (400 ng/mL). The OD measured in the presence of the streptavidin-HRP alone was shown.

FIG. 6: Cross-reactivity against cynomolgus Tyro3 ECD of anti-Tyro3 in the scFv (FIG. 6A) and IgG format (FIG. 6B).

FIG. 7: Cross-reactivity against murine Tyro3 ECD of anti-Tyro3 in the scFv format.

FIG. 8: Epitope restriction of anti-Tyro3 scFvs to structural domains of Tyro3 ECD.

FIG. 9: Binding of anti-Tyro3 IgGs in living cells. HEK293 cells transfected to express recombinant human Tyro3 and HEK293 untransfected cells were stained with the Live-Dead NIR dye and either incubated with the TYRO 3-specific MAB859 monoclonal antibody, its isotype control (upper panels) or to individual anti-Tyro3 IgG (middle and bottom panels). IgG isotype control is IGX2656. MAB002 and MAB859 binding was revealed with a PE-conjugated Goat anti-Mouse Ig Ab (BD Pharmingen). Anti-Tyro3 IgG binding was revealed with a PE-conjugated Mouse-anti Human Lambda Light Chain Ab. Data were acquired using an ARIA III flow cytometer (Becton Dickinson) and analyzed using the Kaluza software (Beckman Coulter). Histograms shown are restricted to live cells that show a characteristic size and granulometry of HEK293 cells. Anti-Tyro3 IgG Tyro3-specific binding is shown by fluorescence intensity increase of Tyro3-transfected cells (black line) compared to untransfected cells (grey line).

FIG. 10: Sensorgram traces from interactions between different captured anti-hTYRO3 IgGs of the invention and the extra cellular domain of human Tyro3 protein.

FIG. 11: Kinetic parameters of anti-human Tyro3 IgGs evaluated by Biolayer Interferometry (BLI) on octet K2.

### DETAILED DESCRIPTION OF THE INVENTION

### Definitions

As used herein, the term “Tyro3”, “Tyro3 tyrosine kinase” or “Tyro3 receptor” refers to the Tyro3 protein, preferably to the human Tyro3 protein. Human Tyro3 (Gene ID: 7301) is also known as, Byk, Dtk, Rse, Rek, Sky or Tif, and is a member of the Tyro3/Axl/Mer (TAM) family of receptor tyrosine kinases. The polynucleotide and amino acid sequences are well-known in the art. Reference sequences are Genbank Accession Nos MN_006293.2 and NP_006284.2, respectively. The reference entry for human Tyro3 in the transcriptome database UniGene is Hs.381282. Tyro3 receptor is composed of an N-terminal extracellular domain comprising two immunoglobulin (Ig)-related domains, Ig-1 and Ig-2, and two fibronectin type III (FNIII)-related domains, FNIII-1 and FNIII-2, followed by a single transmembrane helix and a cytoplasmic tyrosine kinase domain. The positions of each of the domains are known and are readily accessible in public databases. The extracellular domain of the human Tyro3 protein, as disclosed in the Uniprot Accession No. Q06418 (SEQ ID NO 1), includes a first Ig domain (Ig-1) from position 41 to position 128 of SEQ ID NO 1, a second Ig domain (Ig-2) from position 139 to position 220 of SEQ ID NO 1, a first FNIII domain (FNIII-1) from position 227 to position 320 of SEQ ID NO 1, and a second FNIII domain (FNIII-2) from position 325 to position 416 of SEQ ID NO 1. The Tyro3 protein-tyrosine kinase domain extends from position 518 to position 790 of SEQ ID NO 1.

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (Ckappa) or lambda (Clambda) light chains. The constant light chain typically comprises a single domain, and as defined herein refers to positions 108-214 of Ckappa, or Clambda, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. A light or heavy chain variable region (VL or VH) consists of a framework region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework region of an antibody, that is the combined framework regions of the constituent light and heavy chains, serves to position and align the CDRs, which are primarily responsible for binding to an antigen.

By “framework” or “FR” region as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). The AbM CDRs represent a compromise between the Kabat CDRs and Chothia structural loops, and are used by Oxford Molecular's AbM antibody modelling software. The “Contact” CDRs are based on an analysis of the available complex crystal structures. The IMGT unique numbering has been defined to compare the variable domains whatever the antigen receptor, the chain type, or the species (Lefranc M.-P., Immunology Today 18, 509 (1997); Lefranc M.-P., The Immunologist, 7, 132-136 (1999); Lefranc, M.-P., et al., Dev. Comp. Immunol., 27, 55-77 (2003)). Typically, the numbering of amino acid residues in this region is performed by the method described in Kabat et al., supra.

The term “Kabat position” or “variable domain residue numbering as in Kabat” or “amino acid position numbering as in Kabat” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. The Kabat numbering of residues may be determined for a given antibody by alignment at regions of homology of the sequence of the antibody with a “standard” Kabat numbered sequence.

As used in this specification, the term “about” refers to a range of values±10% of the specified value, more preferably a range of values±5% of the specified value. For instance, “about 1” means from 0.9 to 1.1 when 10% is considered and from 0.95 to 1.05 when 5% is considered.

As used herein, the term “consisting essentially of” refers to an amino acid sequence having at least 80, 85, 90, 91, 92, 93, 94, 95, 96, 97, 98 or 99% sequence identity with the reference sequence. The sequence may contain substitutions, preferably conservative substitutions, insertions or deletions relative to the reference sequence. Preferably, the sequence may contain 1 to 10 substitutions, insertions or deletions relative to the reference sequence, more preferably 1, 2, 3, 4 or 5, even more preferably one or two, substitutions, insertions or deletions relative to the reference sequence. In certain embodiments, substitutions, insertions and/or deletions occur in regions outside the CDRs (i.e. in the framework regions).

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://www.ncbi.nlm.nih.gov/igblast/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), betabranched side chains (e.g., threonine, valine, isoleucine) and aromatic side chains (e.g., tyrosine, phenylalanine, tryptophan, histidine).

As used herein, the term “purified” or “isolated”, in relation to a polypeptide or nucleic acid, refers to a polypeptide or nucleic acid which is not in its natural medium or form. The term “isolated” thus includes a polypeptide or nucleic acid removed from its original environment, e.g., the natural environment if it is naturally occurring. For instance, an isolated polypeptide is typically devoid of at least some proteins or other constituents of the cells to which it is normally associated or with which it is normally admixed or in solution. An isolated polypeptide includes said polypeptide naturally-produced contained in a cell lysate; the polypeptide in a purified or partially purified form, the recombinant polypeptide, the polypeptide which is expressed or secreted by a cell, as well as the polypeptide in a heterologous host cell or culture. In relation to a nucleic acid, the term isolated or purified indicates e.g., that the nucleic acid is not in its natural genomic context (e.g., in a vector, as an expression cassette, linked to a promoter, or artificially introduced in a heterologous host cell).

Antibodies of the Invention

The present invention is based on the generation of novel antibodies that are therapeutically useful to effectively and selectively modulate the Tyro3 receptor activity in a human subject.

Accordingly, in a first aspect, the present invention related to an antibody that binds to the extracellular domain of human Tyro3 receptor.

The term “antibody” herein is used in the broadest sense and specifically covers monoclonal antibodies (including full length monoclonal antibodies), polyclonal antibodies, multispecific antibodies (e.g., bispecific antibodies), antibody fragments, and derivatives thereof, so long as they exhibit the desired biological activity. In some embodiments, an antibody may be part of a larger fusion molecule, formed by covalent or non-covalent association of the antibody with one or more other proteins or peptides.

In some embodiments, the antibody is a full length antibody. The term “full length antibody”, as used herein, refers to an antibody having a structure substantially similar to a native antibody structure, not antibody fragments as defined below. The terms particularly refer to an antibody with heavy chains that contain an Fc region. In preferred embodiments, the antibody in a full length IgG antibody selected from IgG1, IgG2, IgG3 and IgG4, preferably a full length IgG1 antibody.

In some other embodiments, the antibody is an antibody fragment. “Antibody fragments” comprise a portion of a full length antibody, generally the antigen binding or variable domain thereof. Examples of antibody fragments include Fab, Fab′, F(ab)₂, F(ab′)₂, F(ab)₃, Fv (typically the VL and VH domains of a single arm of an antibody), single-chain Fv (ScFv), dsFv, Fd (typically the VH and CH1 domains) and dAb (typically a VH domain) fragments, minibodies, diabodies, triabodies, tetrabodies, kappa bodies, linear antibodies, single-chain antibody molecules, multispecific antibodies formed from antibody fragments, and other antibody fragments that retain antigen-binding function (e.g. Holliger and Hudson, Nat Biotechnol. 2005 September; 23(9):1126-36). Antibody fragments can be made by various techniques, including but not limited to proteolytic digestion of intact antibody as well as recombinant host cells (e.g. E. coli or phage). These techniques are well-known by the skilled person and are extensively described in the literature.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

By “Fab”, “Fab fragment” or “Fab region” as used herein is meant the polypeptide that comprises the VH, CH1, VL, and CL immunoglobulin domains. Fab may refer to this region in isolation or this region in the context of a polypeptide as described herein.

Fab′ fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known.

By “Fv”, “Fv region” or “Fv fragment” as used herein is meant a polypeptide that comprises the VL and VH domains of a single antibody. This fragment is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. Fv may refer to this region in isolation or this region in the context of a polypeptide as described herein.

“Single-chain Fv” or “scFv” antibody fragments comprise the VH and VL domains of antibody, wherein these domains are present in a single polypeptide chain. For a review of scFv see Pluckthun in The Pharmacology of Monoclonal Antibodies, vol. 113, Rosenburg and Moore eds. Springer-Verlag, New York, pp. 269-315 (1994).

By “Fc”, “Fc fragment” or “Fc region”, used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to stretch from an amino acid residue at position C226, P230 or A231 to the carboxyl-terminus, wherein the numbering is according to the EU index. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Fc may refer to this region in isolation or this region in the context of a polypeptide as described herein.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In some preferred embodiments, the antibody is an antibody fragment selected from the group consisting of Fab′, F(ab)₂, F(ab′)₂, F(ab)₃, Fv, single-chain Fv (ScFv) fragments and diabodies.

The term “antibody derivative”, as used herein, refers to an antibody provided herein, e.g. a full-length antibody or a fragment of an antibody, wherein one or more of the amino acids are chemically modified, e.g. by alkylation, PEGylation, acylation, ester or amide formation or the like. In particular, this term may refer to an antibody provided herein that is further modified to contain additional nonproteinaceous moieties that are known in the art and readily available. The moieties suitable for derivatization of the antibody include but are not limited to water soluble polymers. Examples of water soluble polymers include, but are not limited to, PEG, copolymers of ethylene glycol/propylene glycol, carboxymethylcellulose, dextran and polyvinyl alcohol.

The derivative may also be an immunoconjugate comprising an anti-Tyro3 antibody of the invention conjugated to one or more heterologous molecule(s), including but not limited to a cytotoxic agent, a detectable moiety such as a fluorescent moiety, a diagnostic radioisotope or an imaging agent; or to a solid support, such as agarose beads or the like. Examples of cytotoxic agents include, but are not limited to chemotherapeutic agents or drugs, growth inhibitory agents, toxins (e.g., protein toxins, enzymatically active toxins of bacterial, fungal, plant, or animal origin, or fragments thereof), or radioactive isotopes. Conjugates of an antibody and cytotoxic agent may be made using a variety of bifunctional protein coupling agents well known by the skilled person. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52: 127-131 (1992)) may be used.

The antibody of the invention may comprise a functional Fc region, a native sequence Fc region or a variant Fc region.

A “functional Fc region” possesses an effector function of a native sequence Fc region. Exemplary “effector functions” include C1q binding; Cell Dependent Cytotoxicity (CDC); Fc receptor binding; Antibody Dependent Cell Cytotoxicity (ADCC), phagocytosis, Antibody Dependent Cell Phagocytosis (ADCP), down regulation of cell surface receptors, etc. Such effector functions generally require the Fc region to be combined with a binding domain (e.g., an antibody variable domain) and can be assessed using various well known assays

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

In certain embodiments, the antibody provided herein is a multispecific antibody, e.g. a bispecific antibody. Multispecific antibodies are monoclonal antibodies that have binding specificities for at least two different sites. In certain embodiments, one of the binding specificities is for Tyro3, in particular the extracellular domain of human Tyro3, and the other is for any other antigen. In certain embodiments, bispecific antibodies may bind to two different epitopes of Tyro3. Bispecific antibodies may also be used to localize cytotoxic agents to cells which express Tyro3. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science 229: 81 (1985)); using leucine zippers to produce bi-specific antibodies (see, e.g., Kostelny et al., J. Immunol., 148(5): 1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol 152:5368 (1994)); and preparing trispecific antibodies as described, e.g., in Tutt et al. J. Immunol. 147: 60 (1991). Engineered antibodies with three or more functional antigen binding sites, including “Octopus antibodies,” are also included herein (see, e.g. US 2006/0025576A1).

In preferred embodiments, the antibody is an isolated antibody. An “isolated” antibody is one which has been separated from a component of its natural environment. In particular, the antibody may be purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g. Flatman et al., J. Chromatogr. B 848:79-87 (2007).

The antibody of the invention may be a polyclonal or monoclonal antibody. Preferably, the antibody is a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods. The “monoclonal antibodies” may also be isolated from phage antibody libraries using the techniques described in Clackson et al., Nature 352:624-628 (1991) and Marks et al., J. Mol. Biol. 222:581-597 (1991), for example.

The antibody of the invention may be a chimeric, humanized or human antibody.

“Chimeric” antibodies are antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Morrison et al., Proc. Natl. Acad Sci. USA 81:6851-6855 (1984)). Preferably, at least a portion of the framework of the antibody is a human consensus framework sequence.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also comprises at least a portion of an immunoglobulin constant region (Fc), typically that of a human immunoglobulin (Jones et al., Nature 321:522-525 (1986); Reichmann et al., Nature 332:323.329 (1988); and Presta, Curr. Op. Struct. Biol. 2:593-596 (1992)).

Chimeric or humanized antibodies can be made by various techniques, well-known by the skilled person and extensively described in the literature.

A “human antibody” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology) and replaced by human loci. See also, for example, Li et al., Proc. Natl. Acad. Sci. USA, 103:3557-3562 (2006) regarding human antibodies generated via a human B-cell hybridoma technology.

In preferred embodiments, the antibody of the invention is a human or humanized monoclonal antibody, more preferably a human monoclonal antibody.

The anti-Tyro3 antibody of the invention exhibits high specificity and/or selectivity for Tyro3, in particular human Tyro3.

As used herein, “specific binding” or “specificity” refers to the ability of an antibody to detectably bind an epitope presented on an antigen, such as Tyro3, while having relatively little detectable reactivity with other proteins or structures (such as other TAM receptors). Antibody specificity may be determined by measurement of cross-reactivity using well-known methods such as ELISA binding assays as described in the experimental section. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

“Selective binding” or “selectivity” refers to the preferential binding of a protein to a particular region, target, or peptide as opposed to one or more other biological molecules, structures, cells, tissues, etc. A Tyro3-binding antibody can be selective for a Tyro3 receptor produced in a particular organism (e.g., in a human as opposed to in a murine organism), and/or for a particular portion of a Tyro3 receptor (such as a particular epitope or antigenic determinant region). For example, selectivity can be determined by competitive ELISA or Biacore assays. The difference in affinity/avidity that marks selectivity can be any detectable preference (e.g., a ratio of more than 1:1.1, or more than about 1:5, if detectable, would be suitable, including 1:10, 1:100, 1:1000 or more).

Preferably, the anti-Tyro3 antibody of the invention specifically/selectively binds to human Tyro3. In particular, said antibody does not significantly bind to human Axl and Mer receptors and/or mammalian non-primate Tyro3 such as murine Tyro3, and in particular to the extracellular domains of these receptors.

In some embodiments, the anti-Tyro3 antibody does not significantly bind to the extracellular domains of human Axl and Mer receptors. The sequence of human Axl protein is disclosed in the Uniprot Accession No. P30530 (SEQ ID NO: 6) and the extracellular domain extends from position 26 to position 451 of SEQ ID NO: 6. The sequence of human Mer protein is disclosed in the Uniprot Accession No. Q12866 (SEQ ID NO: 7) and the extracellular domain extends from position 21 to position 505 of SEQ ID NO: 7.

In some embodiments, the anti-Tyro3 antibody does not significantly bind to the extracellular domain of murine Tyro3. The sequence of murine Tyro3 protein is disclosed in the Uniprot Accession No. P55144 (SEQ ID NO: 8) and the extracellular domain extends from position 31 to position 419 of SEQ ID NO: 8.

In preferred embodiment, the anti-Tyro3 antibody does not significantly bind to the extracellular domains of human Axl and Mer receptors and does not bind to the extracellular domain of murine Tyro3.

Preferably, the anti-Tyro3 antibody further binds to the extracellular domain of cynomolgus monkey (or Macaca fascicularis) Tyro3 receptor. The sequence of the extracellular domain of cynomolgus monkey Tyro3 protein is disclosed in SEQ ID NO: 9. In a particular embodiment, the anti-Tyro3 antibody does not significantly bind to the extracellular domains of human Axl and Mer receptors, does not bind to the extracellular domain of murine Tyro3 and binds to the extracellular domain of cynomolgus monkey Tyro3 receptor.

In other embodiments, the anti-Tyro3 antibody does not significantly bind to the extracellular domains of human Axl and Mer receptors but binds to the extracellular domain of murine Tyro3. This antibody may be directed against a fragment of the receptor that is homologous in human and murine Tyro3 such as, for example, the portion of the extracellular domain of Tyro3 extending from position 310 to position 340 of SEQ ID NO: 1.

The anti-Tyro3 antibody may be a competitive inhibitor of the binding of a Tyro3 ligand, i.e. an antibody that binds to the Tyro3 receptor and that significantly reduces or inhibits the binding of a Tyro3 ligand to said receptor, and in particular to the extracellular domain of human Tyro3 receptor. The competitive inhibitor can bind to Tyro3 with a greater affinity than the Tyro3 ligand. Competition assays can be performed using standard techniques in the art (for instance, competitive ELISA or other binding assays).

The Tyro3 ligand may be a GAS6 protein or a protein S, preferably a GAS6 protein, more preferably human GAS6 protein.

GAS6 (growth arrest-specific 6) belongs structurally to the family of plasma vitamin K-dependent proteins. GAS6 has growth factor-like properties through its interaction with receptor tyrosine kinases of the TAM family; Tyro3, AXL and MER. Human GAS6 is a 678 amino acid protein that consists of a gamma-carboxyglutamate-rich domain that mediates binding to phospholipid membranes, four epidermal growth factor-like domains, and two laminin G-like domains. The reference entry for human GAS6 in the transcriptome database UniGene is Hs.646346.

Protein S is an anticoagulant in the blood coagulation cascade. It acts as a co-factor for activated protein C, a protease that degrades Factor V and Factor VIII and thereby inhibits blood coagulation. The reference entry for human protein S in the transcriptome database UniGene is Hs.64016. Gas6 and Protein S exhibit 44% amino acid sequence identity overall, share the same complex multi-domain structure, and are the only two proteins encoded in the mouse and human genomes that display this configuration of domains.

The anti-Tyro3 antibody of the invention may reduce or inhibit the binding of human Gas6 to human Tyro3 receptor by at least 10%, preferably about 20, 25, 30, 35, 40, 45, 50, 55, 60, 65, 70, 75, 80, 85 to about 90% (percent of ligand blocked at saturating levels of antibodies based on competitive ELISA as described in the experimental section). In particular, the anti-Tyro3 antibodies of the invention may reduce or inhibit the binding of human Gas6 to human Tyro3 receptor in a range from about 25% to about 75%, preferably from about 50% to 75% and more preferably from about 60% to about 75%.

The anti-Tyro3 antibodies of the invention may be an inhibitor/antagonist or an agonist of the Tyro3 receptor.

In preferred embodiments, the anti-Tyro3 antibody is an inhibitor of Tyro3, i.e. an antibody that inhibits or reduces at least one activity of the Tyro3 receptor.

“Tyro3 receptor activity” or “Tyro3 activity” includes any biological activity of Tyro3, for instance an activity that is enhanced or induced by the binding of a Tyro3 ligand, e.g. Gas6 or protein S, to a Tyro3 receptor. In particular, Tyro3 receptor has been shown to be involved in controlling cell survival and proliferation, spermatogenesis, immunoregulation and phagocytosis. This receptor has also been identified as a cell entry factor for Ebola and Marburg viruses.

The antibody may inhibit Tyro3 activity for example by reducing or blocking the binding of a Tyro3 ligand (e.g. Gas6) with the extracellular domain of Tyro3, reducing or blocking Tyro3 mediated signal transduction and/or reducing or blocking Tyro3 phosphorylation and/or reducing or blocking the Tyro3 dimerization. The inhibitory activity can be easily assayed by any method known in the art. In particular, this activity can be determined through a binding assay, a competitive binding assay or a phosphorylation or autophosphorylation assay.

In some other embodiments, the anti-Tyro3 antibody is an agonist of Tyro3, i.e. an antibody that stimulates an activity of the Tyro3 receptor. An agonist may for example facilitate the binding of a Tyro3 ligand to the receptor, inducing or facilitating the dimerization of the receptor and/or inducing or facilitating Tyro3 phosphorylation and/or improving Tyro3 mediated signal transduction. The agonist activity can be easily assayed by any method known in the art such as described above.

The anti-Tyro3 antibody of the invention binds to the extracellular domain of human Tyro3 receptor (from position 41 to position 429 of SEQ ID NO: 1). The antibody may bind one or several of the two immunoglobulin-like domains, Ig-1 and Ig-2, and/or one or several of the two fibronectin type III-like domains, FNIII-1 and FNIII-2.

In a preferred embodiment, the antibody binds the immunoglobulin-like domain Ig-1 of human Tyro3 receptor. In particular, such antibody may bind a polypeptide comprising, consisting essentially of, or consisting of the following amino acid sequence:

AGLKLMGAPVKLTVS QGQPVKLNCSVEGMEEPDIQWVKDGAVVQNLDQ LYIPVSEQHWIGFLSLKSVERSDAGRYWCQVEDGGETEISQPVWLT (SEQ ID NO: 2).

In some other embodiments, the antibody binds the immunoglobulin-like domain Ig-1 of human Tyro3 receptor and does not bind the immunoglobulin-like domain Ig-2 of human Tyro3. Preferably, the antibody specifically or selectively binds the immunoglobulin-like domain Ig-1 of human Tyro3 receptor. In particular, such antibody may bind a polypeptide comprising, consisting essentially of, or consisting of the amino acid sequence of SEQ ID NO: 2, and a polypeptide comprising, consisting essentially of, or consisting of the amino acid sequence of SEQ ID NO: 3, and does not bind, or does not significantly bind, a polypeptide comprising, consisting essentially of, or consisting of the amino acid sequence of SEQ ID NO: 4.

In a particular embodiment, the antibody comprises one, two, three, four, five or six, preferably six, CDRs selected from the group consisting of CDR-L1, CDR-L2 and CDR-L3 of the light chain variable region of SEQ ID NO: 10 and CDR-H1, CDR-H2 and CDR-H3 of the heavy chain variable region of SEQ ID NO: 11. Preferably, said antibody binds the immunoglobulin-like domain Ig-1 of human Tyro3 receptor and does not significantly bind the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 10 and/or a heavy chain comprising one, two or three, preferably three, CDRs selected from the group consisting of CDR-H1, CDR-H2 and CDR-H3 of the heavy chain variable region of SEQ ID NO: 11.

Preferably, the antibody comprises a light chain comprising CDR-L1, CDR-L2 and CDR-L3 of the light chain variable region of SEQ ID NO: 10 and a heavy chain comprising CDR-H1, CDR-H2 and CDR-H3 of the heavy chain variable region of SEQ ID NO: 11.

In a more particular embodiment, the antibody comprises the light chain variable region consisting, or consisting essentially, of amino acids

SYELTQPPSVSVSPGQTARITCSGDALPKKYAYWYQQKSGQAPVLVIYED EKRASGIPERFSGSSSGTMATLTISGAQVEDEADYYCYSQERNGNYAVFGGGTK LTVL (SEQ ID NO: 10) and the heavy chain variable region consisting, or consisting essentially, of amino acids

EVQLVQSGADVKKPGASLTISCQASGYTFTDYWISWVRQKPGKGLEWM GQIVPRASRTNYGPSFQGHVTISADRSTATAYLQWRSLEASDTAMYYCVRPYF QTSGGTIPEYYQHWGPGTLVTVSS (SEQ ID NO: 11).

The sequences of the CDRs according to Chothia, AbM, Kabat, Contact and IMGT definition systems, of the variable regions of SEQ ID NO: 10 and 11, are given below.

TABLE 1
The sequences of the CDRs of the variable
regions of SEQ ID NO: 10 and 11

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 10 | | Contact | KKYAYWY | 13 |
| | | IMGT | ALPKKY | 14 |
| | CDR-L2 | Chothia | EDEKRAS | 15 |
| | | AbM | EDEKRAS | 15 |
| | | Kabat | EDEKRAS | 15 |
| | | Contact | LVIYEDEKRA | 16 |
| | | IMGT | EDE | |
| | CDR-L3 | Chothia | YSQERNGNYAV | 17 |
| | | AbM | YSQERNGNYAV | 17 |
| | | Kabat | YSQERNGNYAV | 17 |
| | | Contact | YSQERNGNYA | 18 |
| | | IMGT | YSQERNGNYAV | 17 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 11 | | Contact | TDYWIS | 22 |
| | | IMGT | GYTFTDYW | 23 |
| | CDR-H2 | Chothia | VPRASR | 24 |
| | | AbM | QIVPRASRTN | 25 |
| | | Kabat | QIVPRASRTNYGPSFQG | 26 |
| | | Contact | WMGQIVPRASRTN | 27 |
| | | IMGT | IVPRASRT | 28 |
| | CDR-H3 | Chothia | PYFQTSGGTIPEYYQH | 29 |
| | | AbM | PYFQTSGGTIPEYYQH | 29 |
| | | Kabat | PYFQTSGGTIPEYYQH | 29 |
| | | Contact | VRPYFQTSGGTIPEYYQ | 30 |
| | | IMGT | VRPYFQTSGGTIPEYYQH | 31 |

In another particular embodiment, the antibody comprises one, two, three, four, five or six, preferably six, CDRs selected from the group consisting of CDR-L1, CDR-L2 and CDR-L3 of the light chain variable region of SEQ ID NO: 32 and CDR-H1, CDR-H2 and CDR-H3 of the heavy chain variable region of SEQ ID NO: 33. Preferably, said antibody binds the immunoglobulin-like domain Ig-1 of human Tyro3 receptor and does not significantly bind the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 32 and/or a heavy chain comprising one, two or three, preferably three, CDRs selected from the group consisting of CDR-H1, CDR-H2 and CDR-H3 of the heavy chain variable region of SEQ ID NO: 33.

Preferably, the antibody comprises a light chain comprising CDR-L1, CDR-L2 and CDR-L3 of the light chain variable region of SEQ ID NO: 32 and a heavy chain comprising CDR-H1, CDR-H2 and CDR-H3 of the heavy chain variable region of SEQ ID NO: 33.

In a more particular embodiment, the antibody comprises the light chain variable region consisting, or consisting essentially, of amino acids

SYELTQPPSVSVSPGQTARITCSGDALPKKYAYWYQQKSGQAPVLVIYED WKRASGIPERFSGSSSGTMATLTISGAQVEDEADYYCYSQERKGNYFVFGGGT KLTVL (SEQ ID NO: 32) and the heavy chain variable region consisting, or consisting essentially, of amino acids

EVQLVQSGADVKKPGASLTISCQASGYTFTDYWISWVRQKPGKGLEWM GQINRHASNTMYGPSFQGHVTISADRSTATAYLQWRSLEASDTAMYYCVRSGG ATSGGTIPEYYQHWGPGTLVTVSS (SEQ ID NO: 33).

The sequences of the CDRs according to Chothia, AbM, Kabat, Contact and IMGT definition systems, of the variable regions of SEQ ID NO: 32 and 33, are given below.

TABLE 2
The sequences of the CDRs of the variable
regions of SEQ ID NO: 32 and 33

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 32 | | Contact | KKYAYWY | 13 |
| | | IMGT | ALPKKY | 14 |
| | CDR-L2 | Chothia | EDWKRAS | 34 |
| | | AbM | EDWKRAS | 34 |
| | | Kabat | EDWKRAS | 34 |
| | | Contact | LVIYEDWKRA | 35 |
| | | IMGT | EDW | |
| | CDR-L3 | Chothia | YSQERKGNYFV | 36 |
| | | AbM | YSQERKGNYFV | 36 |
| | | Kabat | YSQERKGNYFV | 36 |
| | | Contact | YSQERKGNYF | 37 |
| | | IMGT | YSQERKGNYFV | 36 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 33 | | Contact | TDYWIS | 22 |
| | | IMGT | GYTFTDYW | 23 |
| | CDR-H2 | Chothia | NRHASN | 38 |
| | | AbM | QINRHASNTM | 39 |
| | | Kabat | QINRHASNTMYGPSFQG | 40 |
| | | Contact | WMGQINRHASNTM | 41 |
| | | IMGT | INRHASNT | 42 |
| | CDR-H3 | Chothia | SGGATSGGTIPEYYQH | 43 |
| | | AbM | SGGATSGGTIPEYYQH | 43 |
| | | Kabat | SGGATSGGTIPEYYQH | 43 |
| | | Contact | VRSGGATSGGTIPEYYQ | 44 |
| | | IMGT | VRSGGATSGGTIPEYYQH | 45 |

In a further particular embodiment, the antibody comprises one, two, three, four, five or six, preferably six, CDRs selected from the group consisting of CDR-L1, CDR-L2 and CDR-L3 of the light chain variable region of SEQ ID NO: 46 and CDR-H1, CDR-H2 and CDR-H3 of the heavy chain variable region of SEQ ID NO: 47. Preferably, said antibody binds the immunoglobulin-like domain Ig-1 of human Tyro3 receptor and does not significantly bind the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 46 and/or a heavy chain comprising one, two or three, preferably three, CDRs selected from the group consisting of CDR-H1, CDR-H2 and CDR-H3 of the heavy chain variable region of SEQ ID NO: 47.

Preferably, the antibody comprises a light chain comprising CDR-L1, CDR-L2 and CDR-L3 of the light chain variable region of SEQ ID NO: 46 and a heavy chain comprising CDR-H1, CDR-H2 and CDR-H3 of the heavy chain variable region of SEQ ID NO: 47.

In a more particular embodiment, the antibody comprises the light chain variable region consisting, or consisting essentially, of amino acids SYELTQPPSVSVSPGQTARITCSGDALPKKYAYWYQQKSGQAPVLVIYED

KKRVSGIPERFSGSSSGTMATLTISGAQVEDEADYYCYSLAVDGNYGVFGGGT KLTVL (SEQ ID NO: 46) and the heavy chain variable region consisting, or consisting essentially, of amino acids

EVQLVQSGADVKKPGASLTISCQASGYTFTDYWISWVRQKPGKGLEWM GAISRNGSTTYYGPSFQGHVTISADRSTATAYLQWRSLEASDTAMYYCVRSGG LTSGGTIPEYYQHWGPGTLVPVSSVD (SEQ ID NO: 47).

The sequences of the CDRs according to Chothia, AbM, Kabat, Contact and IMGT definition systems, of the variable regions of SEQ ID NO: 46 and 47, are given below.

TABLE 3
The sequences of the CDRs of the variable
regions of SEQ ID NO: 46 and 47

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 46 | | Contact | KKYAYWY | 13 |
| | | IMGT | ALPKKY | 14 |
| | CDR-L2 | Chothia | EDKKRVS | 48 |
| | | AbM | EDKKRVS | 48 |
| | | Kabat | EDKKRVS | 48 |
| | | Contact | LVIYEDKKRV | 49 |
| | | IMGT | EDK | |
| | CDR-L3 | Chothia | YSLAVDGNYGV | 50 |
| | | AbM | YSLAVDGNYGV | 50 |
| | | Kabat | YSLAVDGNYGV | 50 |
| | | Contact | YSLAVDGNYG | 51 |
| | | IMGT | YSLAVDGNYGV | 50 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 47 | | Contact | TDYWIS | 22 |
| | | IMGT | GYTFTDYW | 23 |
| | CDR-H2 | Chothia | SRNGST | 52 |
| | | AbM | AISRNGSTTY | 53 |
| | | Kabat | AISRNGSTTYYGPSFQG | 54 |
| | | Contact | WMGAISRNGSTTY | 55 |
| | | IMGT | ISRNGSTT | 56 |
| | CDR-H3 | Chothia | SGGLTSGGTIPEYYQH | 57 |
| | | AbM | SGGLTSGGTIPEYYQH | 57 |
| | | Kabat | SGGLTSGGTIPEYYQH | 57 |
| | | Contact | VRSGGLTSGGTIPEYYQ | 58 |
| | | IMGT | VRSGGLTSGGTIPEYYQH | 59 |

In another particular embodiment, the antibody comprises one, two, three, four, five or six, preferably six, CDRs selected from the group consisting of CDR-L1, CDR-L2 and CDR-L3 of the light chain variable region of SEQ ID NO: 60 and CDR-H1, CDR-H2 and CDR-H3 of the heavy chain variable region of SEQ ID NO: 61. Preferably, said antibody binds the immunoglobulin-like domain Ig-1 of human Tyro3 receptor and does not significantly bind the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 60 and/or a heavy chain comprising one, two or three, preferably three, CDRs selected from the group consisting of CDR-H1, CDR-H2 and CDR-H3 of the heavy chain variable region of SEQ ID NO: 61.

Preferably, the antibody comprises a light chain comprising CDR-L1, CDR-L2 and CDR-L3 of the light chain variable region of SEQ ID NO: 60 and a heavy chain comprising CDR-H1, CDR-H2 and CDR-H3 of the heavy chain variable region of SEQ ID NO: 61.

In a more particular embodiment, the antibody comprises the light chain variable region consisting, or consisting essentially, of amino acids

SYELTQPPSVSVSPGQTARITCSGDALPKKYAYWYQQKSGQAPVLVIYED SKRPSGIPERFSGSSSGTMATLTISGAQVEDEADYYCYSQNPQGNYFVFGGGTK LTVL (SEQ ID NO: 60) and the heavy chain variable region consisting, or consisting essentially, of amino acids

EVQLVQSGADVKKPGASLTISCQASGYTFTDYWISWVRQKPGKGLEWM GVINKYASWTRYGPSFQGHVTISADRSTATAYLQWRSLEASDTAMYYCVRSG GLTSGGTIPEYYQHWGPGTLVTVSS (SEQ ID NO: 61).

The sequences of the CDRs according to Chothia, AbM, Kabat, Contact and IMGT definition systems, of the variable regions of SEQ ID NO: 60 and 61, are given below.

TABLE 4
The sequences of the CDRs of the variable
regions of SEQ ID NO: 60 and 61

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 60 | | Contact | KKYAYWY | 13 |
| | | IMGT | ALPKKY | 14 |
| | CDR-L2 | Chothia | EDSKRPS | 62 |
| | | AbM | EDSKRPS | 62 |
| | | Kabat | EDSKRPS | 62 |
| | | Contact | LVIYEDSKRP | 63 |
| | | IMGT | EDS | |
| | CDR-L3 | Chothia | YSQNPQGNYFV | 64 |
| | | AbM | YSQNPQGNYFV | 64 |
| | | Kabat | YSQNPQGNYFV | 64 |
| | | Contact | YSQNPQGNYF | 65 |
| | | IMGT | YSQNPQGNYFV | 64 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 61 | | Contact | TDYWIS | 22 |
| | | IMGT | GYTFTDYW | 23 |
| | CDR-H2 | Chothia | NKYASW | 66 |
| | | AbM | VINKYASWTR | 67 |
| | | Kabat | VINKYASWTRYGPSFQG | 68 |
| | | Contact | WMGVINKYASWTR | 69 |
| | | IMGT | INKYASWT | 70 |
| | CDR-H3 | Chothia | SGGLTSGGTIPEYYQH | 71 |
| | | AbM | SGGLTSGGTIPEYYQH | 71 |
| | | Kabat | SGGLTSGGTIPEYYQH | 71 |
| | | Contact | VRSGGLTSGGTIPEYYQ | 72 |
| | | IMGT | VRSGGLTSGGTIPEYYQH | 73 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 74 and CDR-H1, CDR-H2 and CDR-H3 of the heavy chain variable region of SEQ ID NO: 75. Preferably, said antibody binds the immunoglobulin-like domain Ig-1 of human Tyro3 receptor and does not significantly bind the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 74 and/or a heavy chain comprising one, two or three, preferably three, CDRs selected from the group consisting of CDR-H1, CDR-H2 and CDR-H3 of the heavy chain variable region of SEQ ID NO: 75.

Preferably, the antibody comprises a light chain comprising CDR-L1, CDR-L2 and CDR-L3 of the light chain variable region of SEQ ID NO: 74 and a heavy chain comprising CDR-H1, CDR-H2 and CDR-H3 of the heavy chain variable region of SEQ ID NO: 75.

In a more particular embodiment, the antibody comprises the light chain variable region consisting, or consisting essentially, of amino acids

SYELTQPPSVSVSPGQTARITCSGDALPKKYAYWYQQKSGQAPVLVIYED NKRGSGIPERFSGSSSGTMATLTISGAQVEDEADYYCYSKSPEGNYMVFGGGTK LTVL (SEQ ID NO: 74) and the heavy chain variable region consisting, or consisting essentially, of amino acids

EVQLVQSGADVKKPGASLTISCQASGYTFTDYWISWVRQKPGKGLEWM GEIFLPPSTTVYGPSFQGHVTISADRSTATAYLQWRSLEASDTAMYYCVRQTRL TSGGTIPEYYQHWGPGTLVTVSS (SEQ ID NO: 75).

The sequences of the CDRs according to Chothia, AbM, Kabat, Contact and IMGT definition systems, of the variable regions of SEQ ID NO: 74 and 75, are given below.

TABLE 5
The sequences of the CDRs of the variable
regions of SEQ ID NO: 74 and 75

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 74 | | Contact | KKYAYWY | 13 |
| | | IMGT | ALPKKY | 14 |
| | CDR-L2 | Chothia | EDNKRGS | 76 |
| | | AbM | EDNKRGS | 76 |
| | | Kabat | EDNKRGS | 76 |
| | | Contact | LVIYEDNKRG | 77 |
| | | IMGT | EDN | |
| | CDR-L3 | Chothia | YSKSPEGNYMV | 78 |
| | | AbM | YSKSPEGNYMV | 78 |
| | | Kabat | YSKSPEGNYMV | 78 |
| | | Contact | YSKSPEGNYM | 79 |
| | | IMGT | YSKSPEGNYMV | 78 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 75 | | Contact | TDYWIS | 22 |
| | | IMGT | GYTFTDYW | 23 |
| | CDR-H2 | Chothia | FLPPST | 80 |
| | | AbM | EIFLPPSTTV | 81 |
| | | Kabat | EIFLPPSTTVYGPSFQG | 82 |
| | | Contact | WMGEIFLPPSTTV | 83 |
| | | IMGT | IFLPPSTT | 84 |
| | CDR-H3 | Chothia | QTRLTSGGTIPEYYQH | 85 |
| | | AbM | QTRLTSGGTIPEYYQH | 85 |
| | | Kabat | QTRLTSGGTIPEYYQH | 85 |
| | | Contact | VRQTRLTSGGTIPEYYQ | 86 |
| | | IMGT | VRQTRLTSGGTIPEYYQH | 87 |

In some embodiments, the antibody binds the immunoglobulin-like domain Ig-1 but does not bind, or does not significantly bind, the immunoglobulin-like domain Ig-2 of human Tyro3, and comprises

a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 10, 32, 46, 60 or 74, preferably of SEQ ID NO: 10, 32, 46 or 60, and

a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 11, 33, 47, 61 or 75, preferably of SEQ ID NO: 11, 33, 47 or 61.

In some embodiments, the antibody binds the immunoglobulin-like domain Ig-1 of human Tyro3 receptor and binds the immunoglobulin-like domain Ig-2 of human Tyro3. In particular, such antibody may bind
a polypeptide comprising, consisting essentially of, or consisting of the amino acid sequence of SEQ ID NO: 2, and
a polypeptide comprising, consisting essentially of, or consisting of the following amino acid sequence:

AGLKLMGAPVKLTVS QGQPVKLNCS VEGMEEPDIQWVKDGAVVQNLDQ LYIPVSEQHWIGFLSLKSVERSDAGRYWCQVEDGGETEISQPVWLTVEGVPFFT VEPKDLAVPPNAPFQLSCEAVGPPEPVTIVWWRGTTKIGGPAPSPSVLNVTGVT QSTMFSCEAHNLKGLASSRTATVHLQ (SEQ ID NO: 3), and
a polypeptide comprising, consisting essentially of, or consisting of the following amino acid sequence:

VPFFTVEPKDLAVPPNAPFQLSCEAVGPPEPVTIVWWRGTTKIGGPAPSPS VLNVTGVTQSTMFSCEAHNLKGLASSRTATVHLQALPAAPFNITVTKLSSSNAS VAWMPGADGRALLQSCTVQVTQAPGGWEVLAVVVPVPPFTCLLRDLVPATNY SLRVRCANALGPSPYADWVPFQTKGLA (SEQ ID NO: 4).

In a particular embodiment, the antibody comprises one, two, three, four, five or six, preferably six, CDRs selected from the group consisting of CDR-L1, CDR-L2 and CDR-L3 of the light chain variable region of SEQ ID NO: 88 and CDR-H1, CDR-H2 and CDR-H3 of the heavy chain variable region of SEQ ID NO: 89. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 88 and/or a heavy chain comprising one, two or three, preferably three, CDRs selected from the group consisting of CDR-H1, CDR-H2 and CDR-H3 of the heavy chain variable region of SEQ ID NO: 89.

Preferably, the antibody comprises a light chain comprising CDR-L1, CDR-L2 and CDR-L3 of the light chain variable region of SEQ ID NO: 88 and a heavy chain comprising CDR-H1, CDR-H2 and CDR-H3 of the heavy chain variable region of SEQ ID NO: 89.

In a more particular embodiment, the antibody comprises the light chain variable region consisting, or consisting essentially, of amino acids

SYELTQPPSVSVSPGQTARITCSGDALPKKYAYWYQQKSGQAPVLVIYED SKRSSGIPERFSGSSSGTMATLTISGAQVEDEADYYCYSANQRGNYMVFGGGT KLTVL (SEQ ID NO: 88) and the heavy chain variable region consisting, or consisting essentially, of amino acids

EVQLVQSGADVKKPGASLTISCQASGYTFTDYWISWVRQKPGKGLEWM GSITRSSSRTHYGPSFQGHVTISADRSTATAYLQWRSLEASDTAMYYCVRGYTH LRTWGPGTLVTVSS (SEQ ID NO: 89).

The sequences of the CDRs according to Chothia, AbM, Kabat, Contact and IMGT definition systems, of the variable regions of SEQ ID NO: 88 and 89, are given below.

TABLE 6
The sequences of the CDRs of the variable
regions of SEQ ID NO: 88 and 89

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID NO: 88 | | Contact | KKYAYWY | 13 |
| | | IMGT | ALPKKY | 14 |
| | CDR-L2 | Chothia | EDSKRSS | 90 |
| | | AbM | EDSKRSS | 90 |
| | | Kabat | EDSKRSS | 90 |
| | | Contact | LVIYEDSKRS | 91 |
| | | IMGT | EDS | |
| | CDR-L3 | Chothia | YSANQRGNYMV | 92 |
| | | AbM | YSANQRGNYMV | 92 |
| | | Kabat | YSANQRGNYMV | 92 |
| | | Contact | YSANQRGNYM | 93 |
| | | IMGT | YSANQRGNYMV | 92 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID NO: 89 | | Contact | TDYWIS | 22 |
| | | IMGT | GYTFTDYW | 23 |
| | CDR-H2 | Chothia | TRSSSR | 94 |
| | | AbM | SITRSSSRTH | 95 |
| | | Kabat | SITRSSSRTHYGPSFQG | 96 |
| | | Contact | WMGSITRSSSRTH | 97 |
| | | IMGT | ITRSSSRT | 98 |
| | CDR-H3 | Chothia | GYTHLRT | 99 |
| | | AbM | GYTHLRT | 99 |
| | | Kabat | GYTHLRT | 99 |
| | | Contact | VRGYTHLR | 100 |
| | | IMGT | VRGYTHLRT | 101 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 102 and CDR-H1, CDR-H2 and CDR-H3 of the heavy chain variable region of SEQ ID NO: 103. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 102 and/or a heavy chain comprising one, two or three, preferably three, CDRs selected from the group consisting of CDR-H1, CDR-H2 and CDR-H3 of the heavy chain variable region of SEQ ID NO: 103.

Preferably, the antibody comprises a light chain comprising CDR-L1, CDR-L2 and CDR-L3 of the light chain variable region of SEQ ID NO: 102 and a heavy chain comprising CDR-H1, CDR-H2 and CDR-H3 of the heavy chain variable region of SEQ ID NO: 103.

In a more particular embodiment, the antibody comprises the light chain variable region consisting, or consisting essentially, of amino acids

SYELTQPPSVSVSPGQTARITCSGDALPKKYAYWYQQKSGQAPVLVIYED AKRVSGIPERFSGSSSGTMATLTISGAQVEDEADYYCYSASANGNYRVFGGGT KLTVL (SEQ ID NO: 102) and the heavy chain variable region consisting, or consisting essentially, of amino acids

EVQLVQSGADVKKPGASLTISCQASGYTFTDYWISWVRQKPGKGLEWM GRIRPLLSHTMYGPSFQGHVTISADRSTATAYLQWRSLEASDTAMYYCVRSWL ATSGGTIPEYYQHWGPGTLVTVSS (SEQ ID NO: 103).

The sequences of the CDRs according to Chothia, AbM, Kabat, Contact and IMGT definition systems, of the variable regions of SEQ ID NO: 102 and 103, are given below.

TABLE 7
The sequences of the CDRs of the variable
regions of SEQ ID NO: 102 and 103

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 102 | | IMGT | ALPKKY | 14 |
| | CDR-L2 | Chothia | EDAKRVS | 104 |
| | | AbM | EDAKRVS | 104 |
| | | Kabat | EDAKRVS | 104 |
| | | Contact | LVIYEDAKRV | 105 |
| | | IMGT | EDA | |
| | CDR-L3 | Chothia | YSASANGNYRV | 106 |
| | | AbM | YSASANGNYRV | 106 |
| | | Kabat | YSASANGNYRV | 106 |
| | | Contact | YSASANGNYR | 107 |
| | | IMGT | YSASANGNYRV | 106 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 103 | | IMGT | GYTFTDYW | 23 |
| | CDR-H2 | Chothia | RPLLSH | 108 |
| | | AbM | RIRPLLSHTM | 109 |
| | | Kabat | RIRPLLSHTMYGPSFQG | 110 |
| | | Contact | WMGRIRPLLSHTM | 111 |
| | | IMGT | IRPLLSHT | 112 |
| | CDR-H3 | Chothia | SWLATSGGTIPEYYQH | 113 |
| | | AbM | SWLATSGGTIPEYYQH | 113 |
| | | Kabat | SWLATSGGTIPEYYQH | 113 |
| | | Contact | VRSWLATSGGTIPEYYQ | 114 |
| | | IMGT | VRSWLATSGGTIPEYYQH | 115 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 116 and CDR-H1, CDR-H2 and CDR-H3 of the heavy chain variable region of SEQ ID NO: 117. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 116 and/or a heavy chain comprising one, two or three, preferably three, CDRs selected from the group consisting of CDR-H1, CDR-H2 and CDR-H3 of the heavy chain variable region of SEQ ID NO: 117.

Preferably, the antibody comprises a light chain comprising CDR-L1, CDR-L2 and CDR-L3 of the light chain variable region of SEQ ID NO: 116 and a heavy chain comprising CDR-H1, CDR-H2 and CDR-H3 of the heavy chain variable region of SEQ ID NO: 117.

In a more particular embodiment, the antibody comprises the light chain variable region consisting, or consisting essentially, of amino acids

SYELTQPPSVSVSPGQTARITCSGDALPKKYAYWYQQKSGQAPVLVIYED DKRLSGIPERFSGSSSGTMATLTISGAQVEDEADYYCYSTEINGNYAVFGGGTK LTVL (SEQ ID NO: 116) and the heavy chain variable region consisting, or consisting essentially, of amino acids

EVQLVQSGADVKKPGASLTISCQASGYTFTDYWISWVRQKPGKGLEWM GRIHTYQSSTRYGPSFQGHVTISADRSTATAYLQWRSLEASDTAMYYCVRHHS TTTANPYSSWGPGTLVTVSS (SEQ ID NO: 117).

The sequences of the CDRs according to Chothia, AbM, Kabat, Contact and IMGT definition systems, of the variable regions of SEQ ID NO: 116 and 117, are given below.

TABLE 8
The sequences of the CDRs of the variable
regions of SEQ ID NO: 116 and 117

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 116 | | IMGT | ALPKKY | 14 |
| | CDR-L2 | Chothia | EDDKRLS | 118 |
| | | AbM | EDDKRLS | 118 |
| | | Kabat | EDDKRLS | 118 |
| | | Contact | LVIYEDDKRL | 119 |
| | | IMGT | EDD | |
| | CDR-L3 | Chothia | YSTEINGNYAV | 120 |
| | | AbM | YSTEINGNYAV | 120 |
| | | Kabat | YSTEINGNYAV | 120 |
| | | Contact | YSTEINGNYA | 121 |
| | | IMGT | YSTEINGNYAV | 120 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 117 | | IMGT | GYTFTDYW | 23 |
| | CDR-H2 | Chothia | HTYQSS | 122 |
| | | AbM | RIHTYQSSTR | 123 |
| | | Kabat | RIHTYQSSTRYGPSFQG | 124 |
| | | Contact | WMGRIHTYQSSTR | 125 |
| | | IMGT | IHTYQSST | 126 |
| | CDR-H3 | Chothia | HHSTTTANPYSS | 127 |
| | | AbM | HHSTTTANPYSS | 127 |
| | | Kabat | HHSTTTANPYSS | 127 |
| | | Contact | VRHHSTTTANPYS | 128 |
| | | IMGT | VRHHSTTTANPYSS | 129 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 130 and CDR-H1, CDR-H2 and CDR-H3 of the heavy chain variable region of SEQ ID NO: 131. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 130 and/or a heavy chain comprising one, two or three, preferably three, CDRs selected from the group consisting of CDR-H1, CDR-H2 and CDR-H3 of the heavy chain variable region of SEQ ID NO: 131.

Preferably, the antibody comprises a light chain comprising CDR-L1, CDR-L2 and CDR-L3 of the light chain variable region of SEQ ID NO: 130 and a heavy chain comprising CDR-H1, CDR-H2 and CDR-H3 of the heavy chain variable region of SEQ ID NO: 131.

In a more particular embodiment, the antibody comprises the light chain variable region consisting, or consisting essentially, of amino acids

SYELTQPPSVSVSPGQTARITCSGDALPKKYAYWYQQKSGQAPVLVIYED KKRISGIPERFSGSSSGTMATLTISGAQVEDEADYYCYSTSTGGNYMVFGGGTK LTVL (SEQ ID NO: 130) and the heavy chain variable region consisting, or consisting essentially, of amino acids

EVQLVQSGADVKKPGASLTISCQASGYTFTDYWISWVRQKPGKGLEWM GQIRGPASATHYGPSFQGHVTISADRSTATAYLQWRSLEASDTAMYYCVRHQQ QTSGGTIPEYYQHWGPGTLVTVSS (SEQ ID NO: 131).

The sequences of the CDRs according to Chothia, AbM, Kabat, Contact and IMGT definition systems, of the variable regions of SEQ ID NO: 130 and 131, are given below.

TABLE 9
The sequences of the CDRs of the variable
regions of SEQ ID NO: 130 and 131

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 130 | | IMGT | ALPKKY | 14 |
| | CDR-L2 | Chothia | EDKKRIS | 132 |
| | | AbM | EDKKRIS | 132 |
| | | Kabat | EDKKRIS | 132 |
| | | Contact | LVIYEDKKRI | 133 |
| | | IMGT | EDK | |
| | CDR-L3 | Chothia | YSTSTGGNYMV | 134 |
| | | AbM | YSTSTGGNYMV | 134 |
| | | Kabat | YSTSTGGNYMV | 134 |
| | | Contact | YSTSTGGNYM | 135 |
| | | IMGT | YSTSTGGNYMV | 134 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 131 | | IMGT | GYTFTDYW | 23 |
| | CDR-H2 | Chothia | RGPASA | 136 |
| | | AbM | QIRGPASATH | 137 |
| | | Kabat | QIRGPASATHYGPSFQG | 138 |
| | | Contact | WMGQIRGPASATH | 139 |
| | | IMGT | IRGPASAT | 140 |
| | CDR-H3 | Chothia | HQQQTSGGTIPEYYQH | 141 |
| | | AbM | HQQQTSGGTIPEYYQH | 141 |
| | | Kabat | HQQQTSGGTIPEYYQH | 141 |
| | | Contact | VRHQQQTSGGTIPEYYQ | 142 |
| | | IMGT | VRHQQQTSGGTIPEYYQH | 143 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 144 and CDR-H1, CDR-H2 and CDR-H3 of the heavy chain variable region of SEQ ID NO: 145. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 144 and/or a heavy chain comprising one, two or three, preferably three, CDRs selected from the group consisting of CDR-H1, CDR-H2 and CDR-H3 of the heavy chain variable region of SEQ ID NO: 145.

Preferably, the antibody comprises a light chain comprising CDR-L1, CDR-L2 and CDR-L3 of the light chain variable region of SEQ ID NO: 144 and a heavy chain comprising CDR-H1, CDR-H2 and CDR-H3 of the heavy chain variable region of SEQ ID NO: 145.

In a more particular embodiment, the antibody comprises the light chain variable region consisting, or consisting essentially, of amino acids

SYELTQPPSVSVSPGQTARITCSGDALPKKYAYWYQQKSGQAPVLVIYED AKRSSGIPERFSGSSSGTMATLTISGAQVEDEADYYCYSLSDSGNYFVFGGGTK LTVL (SEQ ID NO: 144) and the heavy chain variable region consisting, or consisting essentially, of amino acids

EVQLVQSGADVKKPGASLTISCQASGYTFTDYWISWVRQKPGKGLEWM GQIRGQHSSTLYGPSFQGHVTISADRSTATAYLQWRSLEASDTAMYYCVRHGA STSGGTIPEYYQHWGPGTLVTVSS (SEQ ID NO: 145).

The sequences of the CDRs according to Chothia, AbM, Kabat, Contact and IMGT definition systems, of the variable regions of SEQ ID NO: 144 and 145, are given below.

TABLE 10
The sequences of the CDRs of the variable
regions of SEQ ID NO: 144 and 145

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 144 | | IMGT | ALPKKY | 14 |
| | CDR-L2 | Chothia | EDAKRSS | 146 |
| | | AbM | EDAKRSS | 146 |
| | | Kabat | EDAKRSS | 146 |
| | | Contact | LVIYEDAKRS | 147 |
| | | IMGT | EDA | |
| | CDR-L3 | Chothia | YSLSDSGNYFV | 148 |
| | | AbM | YSLSDSGNYFV | 148 |
| | | Kabat | YSLSDSGNYFV | 148 |
| | | Contact | YSLSDSGNYF | 149 |
| | | IMGT | YSLSDSGNYFV | 148 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 145 | | IMGT | GYTFTDYW | 23 |
| | CDR-H2 | Chothia | RGQHSS | 150 |
| | | AbM | QIRGQHSSTL | 151 |
| | | Kabat | QIRGQHSSTLYGPSFQG | 152 |
| | | Contact | WMGQIRGQHSSTL | 153 |
| | | IMGT | IRGQHSST | 154 |
| | CDR-H3 | Chothia | HGASTSGGTIPEYYQH | 155 |
| | | AbM | HGASTSGGTIPEYYQH | 155 |
| | | Kabat | HGASTSGGTIPEYYQH | 155 |
| | | Contact | VRHGASTSGGTIPEYYQ | 156 |
| | | IMGT | VRHGASTSGGTIPEYYQH | 157 |

In a particular embodiment, the antibody comprises one, two, three, four, five or six, preferably six, CDRs selected from the group consisting of CDR-L1, CDR-L2 and CDR-L3 of the light chain variable region of SEQ ID NO: 158 and CDR-H1, CDR-H2 and CDR-H3 of the heavy chain variable region of SEQ ID NO: 159. Preferably, said antibody binds the immunoglobulin-like domain Ig-1 of human Tyro3 receptor and the immunoglobulin-like domain Ig-2 of human Tyro3.

In particular, the antibody may comprise a light chain comprising one, two or three, preferably three, CDRs selected from the group consisting of CDR-L1, CDR-L2 and CDR-L3 of the light chain variable region of SEQ ID NO: 158 and/or a heavy chain comprising one, two or three, preferably three, CDRs selected from the group consisting of CDR-H1, CDR-H2 and CDR-H3 of the heavy chain variable region of SEQ ID NO: 159.

Preferably, the antibody comprises a light chain comprising CDR-L1, CDR-L2 and CDR-L3 of the light chain variable region of SEQ ID NO: 158 and a heavy chain comprising CDR-H1, CDR-H2 and CDR-H3 of the heavy chain variable region of SEQ ID NO: 159.

In a more particular embodiment, the antibody comprises the light chain variable region consisting, or consisting essentially, of amino acids

SYELTQPPSVSVSPGQTARITCSGDALPKKYAYWYQQKSGQAPVLVIYED HKRASGIPERFSGSSSGTMATLTISGAQVEDEADYYCYSISPAGNYGVFGGGTK LTVL (SEQ ID NO: 158) and the heavy chain variable region consisting, or consisting essentially, of amino acids

EVQLVQSGADVKKPGASLTISCQASGYTFTDYWISWVRQKPGKGLEWM GRISRHGSQTYYGPSFQGHVTISADRSTATAYLQWRSLEASDTAMYYCVRAGG QTSGGTIPEYYQHWGPGTLVTVSS (SEQ ID NO: 159).

The sequences of the CDRs according to Chothia, AbM, Kabat, Contact and IMGT definition systems, of the variable regions of SEQ ID NO: 158 and 159, are given below.

TABLE 11
The sequences of the CDRs of the variable
regions of SEQ ID NO: 158 and 159

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 158 | | IMGT | ALPKKY | 14 |
| | CDR-L2 | Chothia | EDHKRAS | 160 |
| | | AbM | EDHKRAS | 160 |
| | | Kabat | EDHKRAS | 160 |
| | | Contact | LVIYEDHKRA | 161 |
| | | IMGT | EDH | |
| | CDR-L3 | Chothia | YSISPAGNYGV | 162 |
| | | AbM | YSISPAGNYGV | 162 |
| | | Kabat | YSISPAGNYGV | 162 |
| | | Contact | YSISPAGNYG | 163 |
| | | IMGT | YSISPAGNYGV | 162 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 159 | | IMGT | GYTFTDYW | 23 |
| | CDR-H2 | Chothia | SRHGSQ | 164 |
| | | AbM | RISRHGSQTY | 165 |
| | | Kabat | RISRHGSQTYYGPSFQG | 166 |
| | | Contact | WMGRISRHGSQTY | 167 |
| | | IMGT | ISRHGSQT | 168 |
| | CDR-H3 | Chothia | AGGQTSGGTIPEYYQH | 169 |
| | | AbM | AGGQTSGGTIPEYYQH | 169 |
| | | Kabat | AGGQTSGGTIPEYYQH | 169 |
| | | Contact | VRAGGQTSGGTIPEYYQ | 170 |
| | | IMGT | VRAGGQTSGGTIPEYYQH | 171 |

In some embodiments, the antibody binds the immunoglobulin-like domain Ig-1 and the immunoglobulin-like domain Ig-2 of human Tyro3 and comprises

a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 88, 102, 116, 130, 144 or 158, preferably of SEQ ID NO: 102, 116, 130, 144 or 158, and

a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 89, 103, 117, 131, 145 or 159, preferably of SEQ ID NO: 103, 117, 131, 145 or 159.

In other embodiments, the antibody binds the fibronectin III domain FNIII-1 of human Tyro3 receptor. In particular, such antibody may bind
a polypeptide comprising, consisting essentially of, or consisting of the amino acid sequence of SEQ ID NO: 4, and
a polypeptide comprising, consisting essentially of, or consisting of the following amino acid sequence:

AAPFNITVTKLSSSNASVAWMPGADGRALLQSCTVQVTQAPGGWEVLAV VVPVPPFTCLLRDLVPATNYSLRVRCANALGPSPYADWVPFQTKGLAPASAPQ NLHAIRTDSGLILEWEEVIPEAPLEGPLGPYKLSWVQDNGTQDELTVEGTRANL TGWDPQKDLIVRVCVSNAVGCGPWSQPLVVSSHDR (SEQ ID NO: 5),

and does not bind a polypeptide comprising, consisting essentially of, or consisting of the amino acid sequence of SEQ ID NO: 2 or 3.

In a particular embodiment, the antibody comprises one, two, three, four, five or six, preferably six, CDRs selected from the group consisting of CDR-L1, CDR-L2 and CDR-L3 of the light chain variable region of SEQ ID NO: 172 and CDR-H1, CDR-H2 and CDR-H3 of the heavy chain variable region of SEQ ID NO: 173. Preferably, said antibody binds the fibronectin III domain FNIII-1 of human Tyro3 receptor.

In particular, the antibody may comprise a light chain comprising one, two or three, preferably three, CDRs selected from the group consisting of CDR-L1, CDR-L2 and CDR-L3 of the light chain variable region of SEQ ID NO: 172 and/or a heavy chain comprising one, two or three, preferably three, CDRs selected from the group consisting of CDR-H1, CDR-H2 and CDR-H3 of the heavy chain variable region of SEQ ID NO:

173.

Preferably, the antibody comprises a light chain comprising CDR-L1, CDR-L2 and CDR-L3 of the light chain variable region of SEQ ID NO: 172 and a heavy chain comprising CDR-H1, CDR-H2 and CDR-H3 of the heavy chain variable region of SEQ ID NO: 173.

In a more particular embodiment, the antibody comprises the light chain variable region consisting, or consisting essentially, of amino acids

SYELTQPPSVSVSPGQTARITCSGDALPKKYAYWYQQKSGQAPVLVIYED TKRHSGIPERFSGSSSGTMATLTISGAQVEDEADYYCYSAEHRGNYAVFGGGTK LTVL (SEQ ID NO: 172) and the heavy chain variable region consisting, or consisting essentially, of amino acids

EVQLVQSGADVKKPGASLTISCQASGYTFTDYWISWVRQKPGKGLEWM GHINPMRSTTTYGPSFQGHVTISADRSTATAYLQWHSLEASDTAMYYCVRNDN HDDYTYTDDWGPGTLVTVSS (SEQ ID NO: 173).

The sequences of the CDRs according to Chothia, AbM, Kabat, Contact and IMGT definition systems, of the variable regions of SEQ ID NO: 172 and 173, are given below.

TABLE 12
The sequences of the CDRs of the variable
regions of SEQ ID NO: 172 and 173

| Variable | | | | SEQ |
|---|---|---|---|---|
| region | CDR | Definition | Sequence | ID NO |
| Light chain | CDR-L1 | Chothia | SGDALPKKYAY | 12 |
| variable | | AbM | SGDALPKKYAY | 12 |
| region of | | Kabat | SGDALPKKYAY | 12 |
| SEQ ID | | Contact | KKYAYWY | 13 |
| NO: 172 | | IMGT | ALPKKY | 14 |
| | CDR-L2 | Chothia | EDTKRHS | 174 |
| | | AbM | EDTKRHS | 174 |
| | | Kabat | EDTKRHS | 174 |
| | | Contact | LVIYEDTKRH | 175 |
| | | IMGT | EDT | |
| | CDR-L3 | Chothia | YSAEHRGNYAV | 176 |
| | | AbM | YSAEHRGNYAV | 176 |
| | | Kabat | YSAEHRGNYAV | 176 |
| | | Contact | YSAEHRGNYA | 177 |
| | | IMGT | YSAEHRGNYAV | 176 |
| Heavy chain | CDR-H1 | Chothia | GYTFTDY | 19 |
| variable | | AbM | GYTFTDYWIS | 20 |
| region of | | Kabat | DYWIS | 21 |
| SEQ ID | | Contact | TDYWIS | 22 |
| NO: 173 | | IMGT | GYTFTDYW | 23 |
| | CDR-H2 | Chothia | NPMRST | 178 |
| | | AbM | HINPMRSTTT | 179 |
| | | Kabat | HINPMRSTTTYGPSFQG | 180 |
| | | Contact | WMGHINPMRSTTT | 181 |
| | | IMGT | INPMRSTT | 182 |
| | CDR-H3 | Chothia | NDNHDDYTYTDD | 183 |
| | | AbM | NDNHDDYTYTDD | 183 |
| | | Kabat | NDNHDDYTYTDD | 183 |
| | | Contact | VRNDNHDDYTYTD | 184 |
| | | IMGT | VRNDNHDDYTYTDD | 185 |

In preferred embodiments, the antibody comprises a CDR-L1 consisting, or consisting essentially, of the CDR-L1 of the light chain variable region of SEQ ID NO: 10 and/or comprises a CDR-H1 consisting, or consisting essentially, of the CDR-H1 of the heavy chain variable region of SEQ ID NO: 11. Preferably the antibody comprises a CDR-L1 consisting of the CDR-L1 of the light chain variable region of SEQ ID NO: 10 and comprises a CDR-H1 consisting of the CDR-H1 of the heavy chain variable region of SEQ ID NO: 11.

In more particular preferred embodiments, the antibody comprises one, two, three, four, five or six, preferably six, of the following CDRs according to Kabat definition:
a CDR-L1 consisting, or consisting essentially, of the amino acid consensus sequence SGDALPKKYAY (SEQ ID NO: 12);
a CDR-L2 consisting, or consisting essentially, of the amino acid consensus EDX₁RX₂ (SEQ ID NO: 186) wherein X₁ is E, W, K, S, N, A, D, H or T, preferably E, W, K, S, N, A, D or H, and X₂ is A, V, P, G, S, V, L, I or H, preferably A, V, P, G, S, V, L or I;
a CDR-L3 consisting, or consisting essentially, of the amino acid consensus YSX₁X₂X₃X₄GNYX₅V (SEQ ID NO: 187) wherein X₁ is Q, L, K, A, T, L or I, X₂ is E, A, N or S, X₃ is R, V, P, Q, A, I, T, D or H, preferably R, V, P, Q, A, I, T or D, X₄ is N, K, D, Q, E, R, G, S or A, and X₅ is A, F, G, M or R;
a CDR-H1 consisting, or consisting essentially, of the amino acid consensus sequence DYWIS (SEQ ID NO: 21);
a CDR-H2 consisting, or consisting essentially, of the amino acid consensus X₁IX₂X₃X₄X₅SX₆TX₇YGPSFQG (SEQ ID NO: 188) wherein X₁ is Q, A, V, E, S, R or H, preferably Q, A, V, E, S or R, X₂ is V, N, S, F, T, R or H, X₃ is P, R, K, L, T or G, X₄ is R, H, N, Y, P, S, L, Q or M, preferably R, H, N, Y, P, S, L or Q, X₅ is A, G, P, S, L, Q H or R, preferably A, G, P, S, L, Q or H, X₆ is R, N, T, W, H, S, A or Q, and X₇ is N, M, Y, R, V, H, L or T, preferably N, M, Y, R, V, H or L; and/or
a CDR-H3 consisting, or consisting essentially, of the amino acid consensus X₁X₂X₃X₄TSGGTIPEYYQH (SEQ ID NO: 189) wherein X₁ is P, S, Q, H or A, X₂ is Y, G, T, W or Q, X₃ is F, G, R, L, Q or A, and X₄ is Q, A, L or S, or a CDR-H3 consisting, or consisting essentially, of one of the following amino acid sequence: GYTHLRT (SEQ ID NO: 99), HHSTTTANPYSS (SEQ ID NO: 127) or NDNHDDYTYTDD (SEQ ID NO: 183), preferably a CDR-H3 consisting, or consisting essentially, of the amino acid consensus X₁X₂X₃X₄TSGGTIPEYYQH (SEQ ID NO: 189) as defined above.

The antibodies of the invention can comprise any suitable framework variable domain sequence, provided binding activity to Tyro3 is substantially retained. In particular embodiments, the variable domain framework of the antibody is as defined in SEQ ID NO: 10 and 11 by the regions separating the CDRs (FR1, CDR1, FR2, CDR2, FR3, CDR3 and FR4).

In preferred embodiments, the antibody is a full length IgG antibody, i.e. IgG1, IgG2, IgG3 or IgG4, preferably IgG1 antibody. The antibody may comprise any of the variable regions of light and heavy chains as described above, a constant human IgG chain, preferably IgG1 chain, and human lambda or kappa chain, preferably lambda chain. An exemplary sequence of constant human IgG1 chain is set forth in SEQ ID NO: 190. An exemplary sequence of constant human lambda chain is set forth in SEQ ID NO: 191.

In preferred embodiments, the antibody binds human Tyro3 receptor with high affinity. “Binding affinity” generally refers to the strength of the sum of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

In certain embodiments, an antibody provided herein has a dissociation constant (Kd) of 10⁻⁵ M or lower, preferably a Kd of 9, 8, 7, 6, 5, 4, 3, 2 or 1·10⁻⁶ M or lower, more preferably a Kd of 9, 8, 7, 6, 5, 4, 3, 2 or 1·10⁻⁷ M or lower and even more preferably a Kd of 9, 8, 7, 6, 5, 4, 3, 2 or 1·10⁻⁸ M or lower. In most preferred embodiments, the antibody provided herein has a dissociation constant 9, 8, 7, 6, 5, 4, 3, 2 or 1·10⁻⁹ M or lower.

In a particular embodiment, the antibody of the invention has a dissociation constant (Kd) of 3.1×10⁻⁷ M or lower and is preferably selected from the group of antibodies having the variable domains of the heavy and light chains or the CDR sequences of ScFv 2410, 2412, 2413, 2414, 2415, 2710, 2711 or 2713 described in tables 13 and 14. In another embodiment, the antibody of the invention has a dissociation constant (Kd) of 1.4×10⁻⁷ M or lower and is preferably selected from the group of antibodies having the variable domains of the heavy and light chains or the CDR sequences of ScFv 2410, 2412, 2413, 2415, 2710, or 2713 described in tables 13 and 14. In a further embodiment, the antibody of the invention has a dissociation constant (Kd) of 7.4×10⁻⁸ M or lower and is preferably selected from the group of antibodies having the variable domains of the heavy and light chains or the CDR sequences of ScFv 2410, 2415, 2710, or 2713 described in tables 13 and 14. In a further embodiment, the antibody of the invention has a dissociation constant (Kd) of 4×10⁻⁸ M or lower and is preferably an antibody having the variable domains of the heavy and light chains or the CDR sequences of ScFv 2713 described in tables 13 and 14.

Kd may be measured using any technique known by the skilled person. In particular, to measure binding affinity between anti-Tyro3 antibodies and human Tyro3 ECD (positions 40-429 of SEQ ID NO: 1), surface plasmon resonance (SPR) measurements with a Biacore T100 (GE Healthcare, Uppsala, Sweden) may be performed on Series S CMS sensor chips coated with Affibody® (AFFIBODY AB reference cat 10.0623.02.0005). The capture of IgGs to have a maximum response around 100 RU may be achieved using a 15s to 55s injection time. For kinetic measurements, solutions of human Tyro3 ECD from 0.15 to 10 μM may be injected in PBS-P buffer at 25° C. with a flow rate of 30 μl/min. The equilibrium dissociation constants (KD) can be calculated using the heterogeneous ligand model and 1;1 model (BIAcore Evaluation Software version 3.2). The equilibrium dissociation constant (Kd) is calculated as the ratio koff/kon See, e.g., Chen et al., J. Mol. Biol. 293:865-881 (1999). Kd, koff (or Kdis) and kon constants may be also measured by biolayer interferometry (BLI) as described below in the experimental section (cf. example 5).

In some particular embodiments, the antibody of the invention has a kon constant of 1×10⁴ M⁻¹s⁻¹ or higher, preferably of 2, 3, 4, 5, 6, 7, 8, 9×10⁴ M⁻¹s⁻¹ or higher. More preferably, the antibody of the invention has a kon constant of 1×10⁵ M⁻¹s⁻¹ or higher, preferably of 1.2×10⁵ M⁻¹s⁻¹ or higher, and is preferably selected from the group of antibodies having the variable domains of the heavy and light chains or the CDR sequences of ScFv 2410 or 2713 described in tables 13 and 14.

In some particular embodiments, the antibody of the invention has a koff constant of 1×10⁻²s⁻¹ or lower, preferably of 9, 8, 7, 6, 5, 4×10⁻³s⁻¹ or lower. More preferably, the antibody of the invention has a koff constant of 3×10⁻³s⁻¹ or lower and is preferably an antibody having the variable domains of the heavy and light chains or the CDR sequences of ScFv 2710 described in tables 13 and 14.

In some embodiments, the invention provides a human or humanized antibody that competes with an antibody as described above, and more particularly with an antibody selected from the group consisting of the antibodies described in table 13, i.e. antibodies 2410, 2414, 2709, 2710, 2717, 2411, 2412, 2413, 2415, 2711, 2713 and 2718, in the binding to human Tyro3. An antibody competes with another if it binds essentially to the same epitope. Protocols based on ELISA, radioimmunological assays, western blot analyses, or the use of a BIACORE analysis are suitable for use in simple competition tests. The surface in the simple competition assay is preferably a BIACORE chip (or another support compatible with surface plasmon resonance analysis). The control antibody (for example, one of the antibodies described in Table 13) is contacted with a surface at a saturating concentration of the antigen and binding of the control antibody to the surface carrying the antigen is measured. Said binding of the control antibody is compared with the binding of the control antibody to the surface carrying the antigen in the presence of the candidate antibody. In an assay test, a significant reduction in binding of the control antibody in the presence of the candidate antibody indicates that the candidate antibody essentially recognizes the same epitope as the control antibody and thus competes with this antibody. Any candidate antibody which reduces the binding of the control antibody by at least about 30, 40 or 50%, preferably at least about 60, 70% or more, can be considered as an antibody which competes with the control antibody. It shall be understood that the order between the control and candidate antibodies can be reversed, that is to say, that the control antibody can be the first to bind to the surface and that the candidate antibody is subsequently contacted with the surface in a competition test.

In certain embodiments, the antibody of the invention is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (see e.g., Wright et al. TIBTECH, 1997, 15:26-32). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%). The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Examples of publications related to “defucosylated” or “fucose-deficient” antibody variants include, but are not limited to, Okazaki et al. J. Mol. Biol. 336: 1239-1249 (2004) and Yamane-Ohnuki N, Satoh M. mAbs. 2009; 1:230-236. Examples of cell lines capable of producing defucosylated antibodies include Led 3 CHO cells deficient in protein fucosylation (Ripka et al. Arch. Biochem. Biophys. 249:533-545 (1986)), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006)).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

Nucleic Acids, Vectors, Host Cells and Recombinant Production

In another aspect, the present invention concerns a nucleic acid encoding an antibody of the invention as described above, or a nucleic acid complementary to said encoding sequence. Preferably, the nucleic acid is an isolated or purified nucleic acid.

The nucleic acid can be DNA (cDNA or gDNA), RNA, or a mixture of the two. It can be in single stranded form or in duplex form or a mixture of the two. It can comprise modified nucleotides, comprising for example a modified bond, a modified purine or pyrimidine base, or a modified sugar. It can be prepared by any method known to one skilled in the art, including chemical synthesis, recombination, and mutagenesis. The nucleic acid according to the invention may be deduced from the sequence of the antibody according to the invention and codon usage may be adapted according to the host cell in which the nucleic acid shall be transcribed. These steps may be carried out according to methods well known to one of skill in the art and some of which are described in the reference manual Sambrook et al. (Sambrook J, Russell D (2001) Molecular cloning: a laboratory manual, Third Edition Cold Spring Harbor).

The nucleic acid of the invention may encode an amino acid sequence comprising the light chain and/or an amino acid sequence comprising the heavy chain of the antibody, or may be complementary to such encoding sequence.

The present invention further provides a vector comprising a nucleic acid of the invention. Optionally, the vector may comprise several nucleic acids of the invention. In particular, the vector may comprise a nucleic acid of the invention operably linked to a regulatory region, i.e. a region comprising one or more control sequences. Optionally, the vector may comprise several nucleic acids of the invention operably linked to several regulatory regions.

The term “control sequences” means nucleic acid sequences necessary for expression of a coding region. Control sequences may be endogenous or heterologous. Well-known control sequences and currently used by the person skilled in the art will be preferred. Such control sequences include, but are not limited to, promoter, signal peptide sequence and transcription terminator.

The term “operably linked” means a configuration in which a control sequence is placed at an appropriate position relative to a coding sequence, in such a way that the control sequence directs expression of the coding region.

The present invention further relates to the use of a nucleic acid or vector according to the invention to transform, transfect or transduce a host cell.

The present invention also provides a host cell comprising one or several nucleic acids of the invention and/or one or several vectors of the invention.

The term “host cell” also encompasses any progeny of a parent host cell that is not identical to the parent host cell due to mutations that occur during replication.

Suitable host cells for cloning or expression of antibody-encoding vectors include prokaryotic or eukaryotic cells described herein.

For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell lysate in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

The present invention also concerns a method for producing an antibody of the invention, comprising culturing a host cell comprising a nucleic acid of the invention or a vector of the invention, as provided above, under conditions suitable for expression of the antibody, and optionally recovering the antibody from the host cell or from the host cell culture medium. Optionally, the recovered antibody may be further purified or isolated. Suitable media, culture conditions and production method are well-known by the skilled person and can be easily chosen according to the host cell and the antibody to be produced.

Pharmaceutical Compositions

The present invention further relates to a pharmaceutical composition comprising an antibody, a nucleic acid, a vector or a host cell of the invention. The composition may comprise one or several antibodies of the invention, one or several nucleic acid of the invention and/or one or several vectors of the invention and/or one or several host cells of the invention. Preferably, the pharmaceutical composition comprises one or several antibodies of the invention.

Pharmaceutical compositions comprising an antibody of the invention can be formulated according to known methods to prepare pharmaceutically useful compositions, whereby the antibody having the desired degree of purity is mixed with optional physiologically acceptable carriers, excipients or stabilizers (Remington: The Science and Practice of Pharmacy 20th edition (2000)), in the form of aqueous solutions, lyophilized or other dried formulations.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. Preferably, such formulations are sterile, i.e. aseptic or free from all living microorganisms and their spores.

Acceptable carriers, excipients, or stabilizers are nontoxic to recipients at the dosages and concentrations employed, and include, but are not limited to, buffering agents, stabilizing agents, preservatives, isotonifiers, non-ionic detergents, antioxidants and other miscellaneous additives.

Buffering agents help to maintain the pH in the range which approximates physiological conditions. They are preferably present at concentration ranging from about 1 mM to about 50 mM. Suitable buffering agents for use with the present invention include, but are not limited to, both organic and inorganic acids and salts thereof such as citrate, succinate, tartrate, fumarate, gluconate, oxalate, lactate and acetate buffers, as well as phosphate buffers, histidine buffers and trimethylamine salts such as Tris.

Preservatives may be added to retard microbial growth, and may be added in amounts ranging from 0.2%-1% (w/v). Suitable preservatives for use with the present invention include, but are not limited to, phenol, butyl or benzyl alcohol; meta-cresol; alkyl parabens such as methyl or propyl paraben; octadecyldimethylbenzyl ammonium chloride, benzalkonium halides (e.g., chloride, bromide, iodide); hexamethonium or benzethonium chloride; catechol; resorcinol; cyclohexanol; and 3-pentanol.

Isotonifiers may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizing agents refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, .alpha.-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran. Stabilizers may be present in the range from 0.1 to 10,000 weights per part of weight therapeutic agent.

Non-ionic surfactants or detergents (also known as “wetting agents”) may be added to help solubilize the therapeutic agent as well as to protect the therapeutic protein against agitation-induced aggregation. Suitable non-ionic surfactants include, but are not limited to, polysorbates (20, 80, etc.), polyoxamers (184, 188 etc.), PLURONICS™, polyols, polyoxyethylene sorbitan monoethers (TWEEN™-20, TWEEN™-80, etc.). Non-ionic surfactants may be present in a range of about 0.05 mg/ml to about 1.0 mg/ml, preferably about 0.07 mg/ml to about 0.2 mg/ml.

Additional miscellaneous excipients include, but are not limited to, bulking agents, (e.g. starch), chelating agents (e.g. EDTA), antioxidants (e.g., ascorbic acid, methionine, vitamin E), and cosolvents.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington: The Science and Practice of Pharmacy 20th edition (2000).

The form of the pharmaceutical compositions, the route of administration, the dosage and the regimen depend upon the condition to be treated, the severity of the illness, the age, weight, and sex of the patient, etc.

The doses used for the administration can be adapted as a function of various parameters, and in particular as a function of the mode of administration used, of the relevant pathology, or alternatively of the desired duration of treatment.

The pharmaceutical compositions of the invention can be formulated for a topical, oral, parenteral, intranasal, intravenous, intramuscular, subcutaneous or intraocular administration and the like.

Preferably, the pharmaceutical formulation is a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions may be prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the compositions formulated for parenteral administration, such as intravenous or intramuscular injection, other pharmaceutically acceptable forms include, e.g. tablets or other solids for oral administration; time release capsules; and any other form currently used.

The pharmaceutical composition of the invention may comprise one or several antibodies of the invention.

The pharmaceutical composition may further comprise one or several additional active compounds. Examples of additional active compounds include, but are not limited to, chemotherapeutic drug, antibiotics, antiparasitic agents, antifungal agents or antiviral agents.

Examples of chemotherapeutic drugs include, but are not limited to, tamoxifen, aromatase inhibitors, trastuzumab, GnRH-analogues, gemcitabine, docetaxel, paclitaxel, mitomycin, cisplatin, carboplatin, oxaliplatin, doxorubicin, daunorubicin, docetaxel, cyclophosphamide, epirubicin, fluorouracil, methotrexate, mitozantrone, vinblastine, vincristine, vinorelbine, bleomycin, estramustine phosphate or etoposide phosphate.

Examples of antibiotics include, but are not limited to, amoxicillin, clarithromycin, cefuroxime, cephalexin ciprofloxacin, doxycycline, metronidazole, terbinafine, levofloxacin, nitrofurantoin, tetracycline, and azithromycin.

Examples of anti-fungal agents, include, but are not limited to, clotrimazole, butenafine, butoconazole, ciclopirox, clioquinol, clioquinol, clotrimazole, econazole, fluconazole, flucytosine, griseofulvin, haloprogin, itraconazole, ketoconazole, miconazole, naftifine, nystatin, oxiconazole, sulconazole, terbinafine, terconazole, tioconazole, and tolnaftate.

Examples of anti-viral agents, include, but are not limited to, zidovudine, didanosine, zalcitabine, stavudine, lamivudine, abacavir, tenofovir, nevirapine, delavirdine, efavirenz, saquinavir, ritonavir, indinavir, nelfinavir, saquinavir, amprenavir, and lopinavir.

The amount of antibody of the invention which will be effective in the treatment of a particular disorder or condition will depend on the nature of the disorder or condition, and can be determined by standard clinical techniques.

In a preferred embodiment, each dose may range from about 0.1 mg to about 25 mg per kilogram of body weight of antibody, or more preferably, from about 1 mg to about 10 mg per kilogram body weight.

The dosing schedule for administration may vary form once a month to daily depending on a number of clinical factors, including the type of disease, severity of disease, and the subject's sensitivity to the therapeutic agent.

Therapeutic and Diagnostic Applications

The present invention further relates to an antibody of the invention or a pharmaceutical composition of the invention for use in the prevention or treatment of a hyperproliferative disease or an infectious disease.

The present invention relates to the use of an antibody of the invention or a pharmaceutical composition of the invention as a medicament for the treatment of a disease. The invention also relates to the use of an antibody of the invention for the manufacture or preparation of a medicament.

In particular, the present invention relates to a method of treating a hyperproliferative disease in a subject, comprising administering to a subject suffering from hyperproliferative disease an effective amount of the antibody or composition of the invention.

As used herein, the term “subject” or “patient” refers to a mammal, preferably a human being.

The effective amount may be a therapeutically or prophylactically effective amount. A “therapeutically effective amount” refers to an amount effective, at dosages and for periods of time necessary, to achieve the desired therapeutic or prophylactic result. The therapeutically effective amount of an antibody or composition of the invention may vary according to factors such as the disease state, age, sex, and weight of the individual, and the ability of the antibody or composition, to elicit a desired response in the individual. A therapeutically effective amount encompasses an amount in which any toxic or detrimental effects of the antibody or composition are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The terms “hyperproliferative disease” refer to disorders that are associated with some degree of abnormal cell proliferation. In one embodiment, the hyperproliferative disease is cancer.

The term “cancer” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include, but are not limited to, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, osteosarcoma, melanoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer. In preferred embodiment, the cancer is selected from bladder tumor, diffuse large B-Cell lymphoma, adenoid cystic carcinoma of salivary gland, Burkitt lymphoma, multiple myeloma, pancreatic ductal adenocarcinoma, hairy cell leukemia, metastactic prostate cancer, melanoma, colorectal cancer.

The present invention also relates to a method of treating a subject affected with an infectious disease comprising administering to the subject an effective amount of the antibody or composition.

The subject may be caused by a pathogen which may be, for example, a virus, a bacterium, a fungus or a parasite.

Examples of infectious bacteria that can be treated with the methods provided herein include, but are not limited to, any type of Gram-positive (such as Streptococcus, Staphylococcus, Corynebacterium, Listeria, Bacillus and Clostridium) or Gram-negative bacteria (such as Salmonella, Shigella, Enterobacteriaceae, Pseudomonas, Moraxella, Helicobacter, Stenotrophomonas, Bdellovibrio, acetic acid bacteria, alpha-proteobacteria, Escherichia coli, Neisseria gonorrhoeae, Neisseria meningitidis, Moraxella catarrhalis, Hemophilus influenzae, Klebsiella pneumoniae, Legionella pneumophila, Pseudomonas aeruginosa, Proteus mirabilis, Enterobacter cloacae and Serratia marcescens. Exemplary infectious bacteria include, but are not limited to: Helicobacter pyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, and Actinomyces israelli.

Examples of infectious fungi that can be treated with the methods provided herein include, but are not limited to, Cryptococcus neoformans, Histoplasma capsulatum, Coccidioides immitis, Blastomyces dermatitidis, Chlamydia trachomatis, and Candida albicans.

Examples of infectious parasites that can be treated with the methods provided herein include, but are not limited to Plasmodium falciparum and Toxoplasma gondii.

Examples of viruses that can be treated with the methods provided herein include, but are not limited to, enveloped viruses such as members of the following viral families: Retroviridae (e.g., HIV (such as HIV1 and HIV2), MLV, SIV, FIV, Human T-cell leukemia viruses 1 and 2, XMRV, and Coltiviruses (such as CTFV or Banna virus)); Togaviridae (for example, alphaviruses (such as Ross River virus, Sindbis virus, Semliki Forest Virus, O'nyong'nyong virus, Chikungunya virus, Eastern equine encephalitis virus, Western equine encephalitis virus, Venezuelan equine encephalitis virus) or rubella viruses); Flaviridae (for example, dengue viruses, encephalitis viruses (such as West Nile virus or Japanese encephalitis virus), yellow fever viruses); Coronaviridae (for example, coronaviruses such as SARS virus or Toroviruses); Rhabdoviridae (for example, vesicular stomatitis viruses, rabies viruses); Paramyxoviridae (for example, parainfluenza viruses, mumps virus, measles virus, respiratory syncytial virus, sendai virus, and metopneumovirus); Orthomyxoviridae (for example, influenza viruses); Bunyaviridae (for example, Hantaan virus, bunya viruses (such as La Crosse virus), phleboviruses, and Nairo viruses); Hepadnaviridae (Hepatitis B viruses); Herpesviridae (herpes simplex virus (HSV) 1 and HSV-2, varicella zoster virus, cytomegalovirus (CMV), HHV-8, HHV-6, HHV-7, and pseudorabies virus); Filoviridae (filoviruses including Ebola virus and Marburg virus) and Poxyiridae (variola viruses, vaccinia viruses, pox viruses (such as small pox, monkey pox, and Molluscum contagiosum virus), yatabox virus (such as Tanapox and Yabapox)). Non-enveloped viruses can also be treated with the methods provided herein, such as members of the following families: Calciviridae (such as strains that cause gastroenteritis); Arenaviridae (hemorrhagic fever viruses such as LCMV, Lassa, Junin, Machupo and Guanarito viruses); Reoviridae (for instance, reoviruses, orbiviruses and rotaviruses); Birnaviridae; Parvoviridae (parvoviruses, such as Human bocavirus adeno-associated virus); Papillomaviridae (such as papillomaviruses); Papovaviridae (papilloma viruses, polyoma viruses); Adenoviridae (adenoviruses); Picornaviridae (enteroviruses, enteric viruses, Poliovirus, coxsackieviruses, hepatoviruses, cardioviruses, aptoviruses, echoviruses, hepatitis A virus, Foot and mouth disease virus, and rhinovirus) and Iridoviridae (such as African swine fever virus). Other viruses that can be treated using the methods provided herein include unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (for instance, Hepatitis C); calciviruses (such as Norovirus, Norwalk and related viruses); Hepeviruses (such as Hepatitis E, JC and BK viruses) and astroviruses).

In the methods of the present invention, the antibody of the invention may be used in combination with other active ingredients that can be chosen according to the disease to be prevented or treated. Examples of other active ingredients include, but are not limited to, chemotherapeutic drugs, antibiotics, antiparasitic agents, antiviral agents or antifungal agents. Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The present invention further relates to an antibody of the invention or a pharmaceutical composition of the invention for use in a method of diagnosis or detection of a disease. The present invention also relates to a method of detecting the presence of Tyro3 in a biological sample.

Any of the anti-Tyro3 antibodies of the invention may be useful for detecting the presence of Tyro3 in a biological sample. The term “detecting” as used herein encompasses quantitative or qualitative detection. In certain embodiments, a biological sample comprises a cell or tissue such as urothelium, breast, pancreas, esophagus, lung or brain.

The method may comprise contacting the biological sample with an antibody of the invention under conditions permissive for binding of the antibody to Tyro3, if present in the sample, and detecting whether a complex is formed between the antibody and Tyro3. Such method may be an in vitro or in vivo method.

Exemplary disorders that may be diagnosed using an antibody of the invention include cancer (for example, cancer of the breast, lung, pancreas, brain, kidney, ovary, stomach, leukemia, uterine endometrium, colon, prostate, thyroid, liver, osteosarcoma, and/or melanoma).

The antibody of the invention may also be used to select subjects eligible for therapy with an anti-Tyro3 antibody, e.g. where Tyro3 is a biomarker for selection of patients.

The invention thus also relates to a method for selecting a subject affected with a disease, in particular a cancer, for a therapy with an anti-Tyro3 antibody or determining whether a subject affected with a disease, in particular a cancer, is susceptible to benefit from a therapy with an anti-Tyro3 antibody.

In some embodiments, the antibody of the invention used in therapeutic or diagnostic methods may be labelled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

The following examples are given for purposes of illustration and not by way of limitation.

### EXAMPLES

### Example 1 Generation of Fully Human Anti-Tyro3 scFvs by Phage Display-Conversion of ScFvs into IgGs

The anti-Tyro3 antibodies, i.e the anti-Tyro3 scFvs, and by conversion the corresponding anti-Tyro3 Immunoglobulins (IgGs), were selected to recognize specifically the extracellular domain of the protein corresponding to the human Tyro3 protein (SEQ ID NO: 1). These anti-Tyro3 scFvs were selected to not recognize other members of the TAM Receptor family, respectively the human AXL (SEQ ID NO: 6) and the human MER protein (SEQ ID NO: 7).

Screening of Anti-Tyro3 ScFvs

ScFvs clones were generated and selected using the Rapid Liquid Screening (RLS) by screening phages displaying specific scFvs for
the extracellular domain (ECD) of the human Tyro3 (hTyro3) protein (from position 41 to position 428 of SEQ ID NO: 1) fused to a human Fc (from R&D Biotech) with non-biotinylated hTyro3 (Recombinant Human Dtk-Fc Chimera, R&D Systems, Minneapolis, Minn.; cat. no. 859-DK)
the bacteria expressed entire hTyro3 ECD (from position 41 to position 430 of SEQ ID NO: 1; AxioMx, cat. no. pAX1635), or
a bacteria expressed peptide (with amino acid sequence DWVPFQTKGLAPASAPQNLHAIRTDSGLIL (SEQ ID NO: 192)) from a 100% homologous region between human and murine Tyro3 ECD and without potential glycosylation sites. This peptide corresponds to the sequence from position 310 to position 340 of SEQ ID NO: 1, at the end of domain FNIII 1 of Tyro3 sequence.

The bacteria expressed proteins were synthesized, cloned, expressed in, and purified, all three proteins as biotinylated maltose-binding protein (MBP) fusions.

Single colonies were screened, positive clones were identified, antibody clones were sequenced, and unique sequences were identified. Positive unique binders were then tested for binding to murine Tyro3 ECD-human Fc (759-DT-100) from R&D Systems.

ScFv Purification and Specificity Testing

For protein production, the lead scFvs were cloned into a protein production vector (AxioMx; cat. no. pAPIII6), and the successfully cloned scFvs were transformed into NEB Turbo Competent E. coli. (New England Biolabs [NEB], Ipswich, Mass.; cat. no. C2984H). Antibodies were expressed in E. coli and purified by affinity chromatography using HisPur Cobalt Resin (Thermo Scientific, cat. no. 89965).

ELISA was performed to assess the ability of the purified proteins to bind to the following ECD-Fc fusions: hTyro3, mTyro3, hAXL (R&D Systems, cat. no. 154-AL-100), and hMER (R&D Systems, cat. no. 891-MR-100). Binding to full-length (FL) hTyro3 from OriGene (OriGene Technologies, Rockville, Md.; cat. no. TP308260) was also assessed.

Once scFvs with the expected criteria were identified, ScFv into IgG conversion as whole IgG1/lambda IgGs and IgG production were done.

ScFv to IgG Conversion

The amino acid sequences of the variable regions of respectively light and heavy chains were deduced from the translation of the selected ScFv nucleotide sequences. The amino acid sequences of the variable regions of light and heavy chain of selected anti Tyro3 ScFvs are presented below. These amino acid sequences constitute the variable regions of corresponding IgG molecules after IgG conversion.

TABLE 13
Amino acid sequences of the variable domains of the heavy and light
chains of selected ScFvs.

| Antibody | VL sequence | VH sequence |
|---|---|---|
| 2410 | SEQ ID NO: 10 | SEQ ID NO: 11 |
| 2414 | SEQ ID NO: 32 | SEQ ID NO: 33 |
| 2709 | SEQ ID NO: 46 | SEQ ID NO: 47 |
| 2710 | SEQ ID NO: 60 | SEQ ID NO: 61 |
| 2717 | SEQ ID NO: 74 | SEQ ID NO: 75 |
| 2411 | SEQ ID NO: 88 | SEQ ID NO: 89 |
| 2412 | SEQ ID NO: 102 | SEQ ID NO: 103 |
| 2413 | SEQ ID NO: 116 | SEQ ID NO: 117 |
| 2415 | SEQ ID NO: 130 | SEQ ID NO: 131 |
| 2711 | SEQ ID NO: 144 | SEQ ID NO: 145 |
| 2713 | SEQ ID NO: 158 | SEQ ID NO: 159 |
| 2718 | SEQ ID NO: 172 | SEQ ID NO: 173 |

The ScFv heavy chains were cloned into pAX 1566 (Invivogen; pFUSE2ss-CHIg-hG1) using EcoRI and NheI cloning sites while scFv light chains were cloned into pAX 1642 (Invivogen; pFUSE2ss-CLIg-h12) using EcoRI and AvrII cloning sites. Sequence-confirmed clones were transformed into NEB 5 alpha cells and transfection grade DNA was prepared using Qiafilter Maxiprep kit (Qiagen, 12263). For mammalian expression, Free-style CHO cells (Life Technologies, R800-07) were co-transfected with heavy and light chain constructs using FreeStyle Max reagent (Life Technologies, 16447-100) according to manufacturer's instructions. Cell supernatants were collected by centrifugation of day 6 post-transfection and loaded on Protein A sepharose column (Life Technologies, 101042). Protein A resin was washed 2× with 10 column volume of PBS-T (PBS/0.1% Tween-20) and eluted with 3 column volumes of Glycine, pH 3.5. The pH was neutralized with 1 M Tris, pH 9.0. IgGs were buffer exchanged into PBS with Amicon Ultra-4 Centrifugal Filters (Millipore, UFC801024). IgGs were analyzed by SDS-PAGE and Coomassie Blue staining and IgG concentration was determined by Coomassie Plus assay (Thermo Scientific, 1856210). Endotoxin levels were measured by LAL Chromogenic assay (Thermo Scientific, 88282).

### Example 2: Definition of the CDR Regions for Each Chain of the Anti Tyro3 IgGs of the Invention

The Complementary Determining regions of the anti-Tyro3 antibodies were defined by submitting light and heavy sequences of the anti Tyro3 IgGs on one hand to the human V quest analysis tool from the IMGT database (http://www.imgt.org/) and, on the other hand, to the request tool from the ABYSIS database (http://www.bioinf.org.uk/abs/) described in Protein Sequence and Structure Analysis of Antibody Variable Domains. In: Antibody Engineering Lab Manual (Ed.: Duebel, S. and Kontermann, R., Springer-Verlag, Heidelberg). The CDRs delimitations results according to IMGT and ABYSIS for each anti-Tyro3 antibody of the invention are summarized in tables 1 to 12, as presented below.

TABLE 14
List of the tables presenting the CDR sequences
of selected anti-Tyro3 antibodies.

| Antibody | CDR sequences presented in: |
|---|---|
| 2410 | Table 1 |
| 2414 | Table 2 |
| 2709 | Table 3 |
| 2710 | Table 4 |
| 2717 | Table 5 |
| 2411 | Table 6 |
| 2412 | Table 7 |
| 2413 | Table 8 |
| 2415 | Table 9 |
| 2711 | Table 10 |
| 2713 | Table 11 |
| 2718 | Table 12 |

### Example 3: Production of Recombinant his-Strep Tagged Tyro3 Extracellular Domain (ECD), and Fragments Thereof

For the different binding assay experiments described herein, the protein sequences corresponding to the entire Tyro3 ECD from human, cynomolgus and murine species were defined based on information provided by the UniProt Knowledge base (UniProtKB) database. The corresponding protein sequence of complete extracellular domain (or ECD) of the human Tyro3 protein corresponds to amino acids 41-429 of SEQ ID NO: 1. For murine Tyro3, the ECD corresponds to amino acids 21-505 of the SEQ ID NO: 8. For cynomolgus, the sequence of the ECD is described in SEQ ID NO: 9.

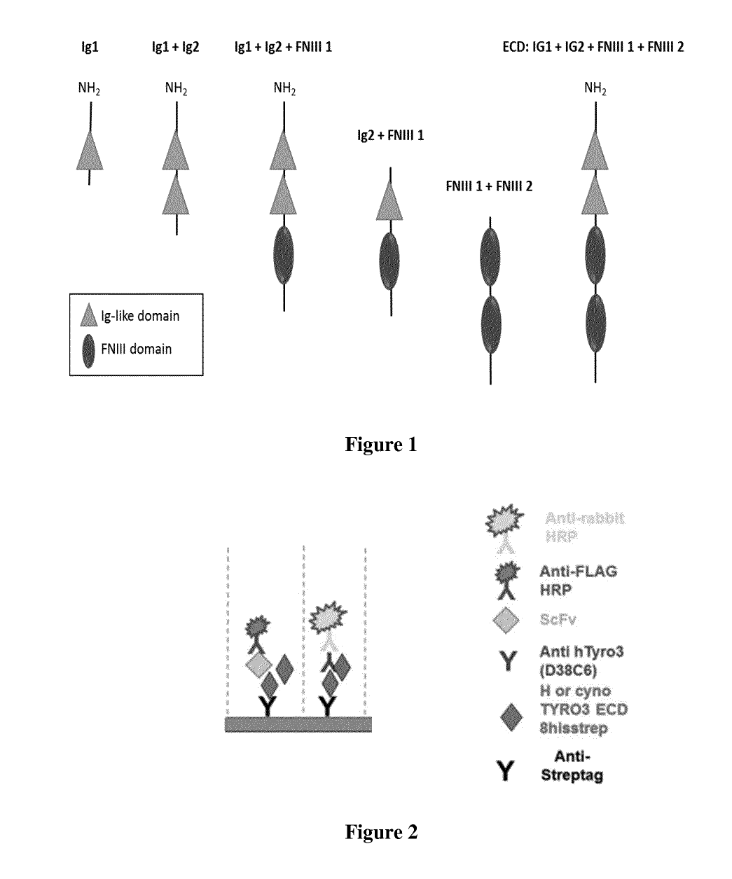

Different fragments of hTyro3 ECD were also produced (FIG. 1). These fragments comprised Ig-1 domain, Ig-1 and Ig-2 domains, Ig-1, Ig-2 and FNIII-1 domains, Ig-2 and FNIII-1 domains, or FNIII-1 and FNIII-2 domains. The amino acid sequences corresponding to Ig-1 domain, Ig-1 and Ig-2 domains, Ig-2 and FNIII-1 domains, and FNIII-1 and FNIII-2 domains are described in SEQ ID NO: 2, 3, 4 and 5, respectively.

The DNA sequences corresponding to these different proteins were codon-optimized for expression in humans, gene-synthetized, assembled and cloned into pCDNA3.3 I-neo (Life Technologies). For all these constructs, the common signal sequence (METDTLLLWVLLLWVPGSTG (SEQ ID NO: 193)) was used for the secretion of recombinant proteins. A C-terminal tag (LVPRGSSAHHHHHHHHSAWSHPQFEK (SEQ ID NO: 194)) consisting of a thrombine cleavage site followed by an octa-histidine (8xHis)-tag coupled to a streptavidin tag, was added in order to facilitate the detection (by anti-histidine or anti-strep tag antibodies), affinity purification or selective immobilization of the proteins onto nitrilotriacetic acid (NTA)/Ni2+ surfaces or onto Strepmab immoplate (IBA Lifesciences).

The human and cynomolgus Tyro3 proteins were transiently transfected in HEK 293 Freestyle cells and purified. The other constructs were successfully expressed as secreted proteins using an in house optimized transient transfection method of mammalian HEK 293 freestyle cells (Life technologies). Briefly, the pcDNA3.3 plasmids encoding each of the constructs were amplified and endotoxin free midi preps (Macherey Nalgene) were prepared. These plasmids were used to electroporate 293 Freestyle cells (life technologies). The proteins were then produced over 7 days at 34° C. in a 8% CO2 shaking incubator in presence of sodium butyrate 1 mM added 24h post electroporation at 32° C. The expressed proteins were separated from the cells by centrifugation 10 min at 8000 rpm and the supernatant was 0.22 μm filtrated before purification. The 8xHis-tagged recombinant Tyro3 proteins expressed in mammalian cells were purified under native conditions using the Ni-NTA Agarose (nitrilotriacetic acid (NTA), a tetradentate chelating ligand, in a highly cross-linked 6% agarose matrix). Proteins bound to the resin are eluted by competition with imidazole. Then, the proteins were further purified and buffer exchanged in PBS IX buffer on preparative HiLoad 26/56 Superdex 200 gel filtration column (GE healthcare). The proteins were 0.22 μm filtrated before −80° c. storage. The protein concentrations were determined using ultraviolet-visible spectroscopy.

The purity, mass and integrity of all purified Tyro3 constructs were checked by SDS PAGE electrophoresis followed by coomassie blue staining and by immuno-blot analysis using a HRP-conjugated streptactin (IBA life sciences). The proteins were also analyzed on Bio SEC-3, (7.8×300) HPLC gel filtration columns, with 150 Å or 300 Å pore sizes according to the size of the constructs to analyze.

### Example 4. ELISA Binding Assay of scFvs/IgGs Against hTyro 3-ECD

ELISA Binding assay was performed to check and compare the capacity of each anti-Tyro3 scFv and of each corresponding converted IgGs to bind human Tyro3 ECD protein (FIG. 2).

The human Tyro3 ECD protein was produced and purified with gel filtration as described in example 3. This protein was produced as a dimeric glycoprotein with an apparent molecular weight of roughly 88 Kda (without glycosylation additional mass).

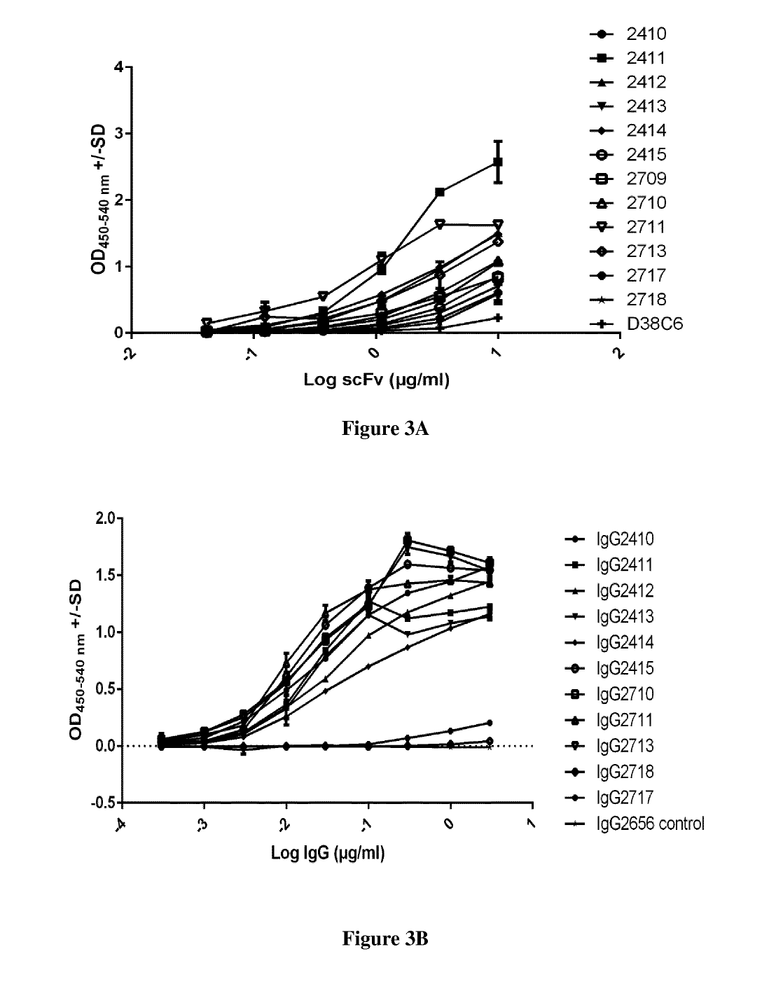

A double sandwich ELISA was developed. This assay consisted in measuring the binding of the anti-Tyro3 antibodies to be tested on immobilized strep-tagged entire human Tyro3 ECD captured directly on ELISA plate pre-coated with an anti-strep mAb (StrepMAB-Immo coated microplates from IBA biotech). Briefly, the 96 wells of StrepMAB-Immo coated microplate were washed three times with 300 μL of the PBS Tween 0.2% (referred as ELISA washing solution). The capture of an excess of purified strep tagged hTyro3 ECD protein was achieved by a one hour incubation at room temperature. In these experiments, 3 μg of purified protein diluted in PBS, BSA 1% (referred as ELISA diluent solution) were immobilized per well. The wells were washed three times with 300 μL of the washing solution. The non-specific binding sites on the well surface were blocked during 60 min at room temperature with 200 μL/well of blocking solution (PBS, BSA 3%). The wells were washed five times with 300 μL of the washing solution. Then, 100 μl (in duplicates) of the different concentrations of anti-Tyro3 antibodies to be tested diluted in ELISA diluent solution, were added per well and allowed to incubate during 1 hour at room temperature. An Irrelevant control IgG2656 was also added in this experiment. For anti-Tyro3 antibodies in the ScFv format, experiments were performed with 6 concentrations in duplicates (FIG. 3A). For anti-Tyro3 antibodies in the IgG format, experiments were performed with 9 concentrations in duplicates (FIG. 3B). The ScFv starting concentration for first dilution was 10 μg/mL (or 30 μg/mL for 2718) and then dilutions of 3 fold in the ELISA diluent solution were performed. For IgGs experiments, starting concentration for first dilution was 3 μg/ml and then dilutions of 3 fold in the ELISA diluent solution were performed. The wells were washed five times with 300 μL of the washing solution. Then, according to the nature of the anti-Tyro3 antibody to be evaluated (see table 15, first column), their binding on the captured Tyro3 construct was revealed by adding 100 μl/well of the corresponding HRP-conjugated secondary antibody described in Table 15 (3^{rd} column) at the indicated dilution (4^{th} column in table 15) diluted in ELISA diluent solution. The mix was allowed to incubated 60 min at room temperature. The wells were washed five times with 300 μL of the washing solution. The revelation of Horseradish Peroxidase activity was realized with 100 μl/well of a ready to use TMB solution and incubated for 5-30 min at room temperature under mixing and without light. Optical density at 450 nm and at 540 nm was read with a Labsystems iEMS MF apparatus.

TABLE 15
ELISA binding assay conditions

| | Initial | | |
|---|---|---|---|
| Primary Antibody | Dilution | Secondary antibody | Dilution |
| Anti-Tyro3 scFv | 10 μg/mL | | |
| Or irrelevant 735 ScFv | or 30 μg/mL | | |
| | for 2718 | | |
| Anti-Tyro3 scFv | | murine HRP | 1/10000 |
| Or irrelevant 735 ScFv | | conjugated anti | |
| | | Flag antibody (M2 | |
| | | from Sigma aldrich) | |
| Anti-Tyro3 IgG or | 10 μg/mL | | |
| Control irrelevant IgG | | | |
| 2656 | | | |
| Anti-Tyro3 IgG or | | goat HRP- | 1/75000 |
| Control irrelevant IgG | | coupled anti-human | |
| 2656 | | IgG1 (kit from | |
| | | Bethyl Laboratories) | |
| N18 goat anti hTyro3 | 1/30 | | |
| (cross reacts with human, | | | |
| mouse and rat Tyro3) | | | |
| N18 goat anti-hTyro3 | | Rabbit anti goat | 1/10000 |
| | | HRP-coupled | |
| | | secondary antibody | |
| D38C6 Rabbit monoclonal | 1/2000 | | |
| anti hTyro3 | | | |
| (cross reacts with murine | | | |
| and cyno Tyro3#5585 Cell | | | |
| Signaling Technology) | | | |
| D38C6 Rabbit anti hTyro3 | | goat anti Rabbit | 1/10000 |
| | | HRP-coupled | |
| | | secondary antibody | |

### Example 5 Affinity Measurement of Anti-Tyro3 IgGs Using Biolayer Interferometry (BLI)

20 mg of the recombinant 8his-strep-tagged the extra cellular domain (ECD) of human Tyro3 (positions 40-429 of SEQ ID NO: 1) were gel filtrated on a Hiload Superdex S200 26/60. Anti humanTyro3 IgGs to be tested were also purified by gel filtration.

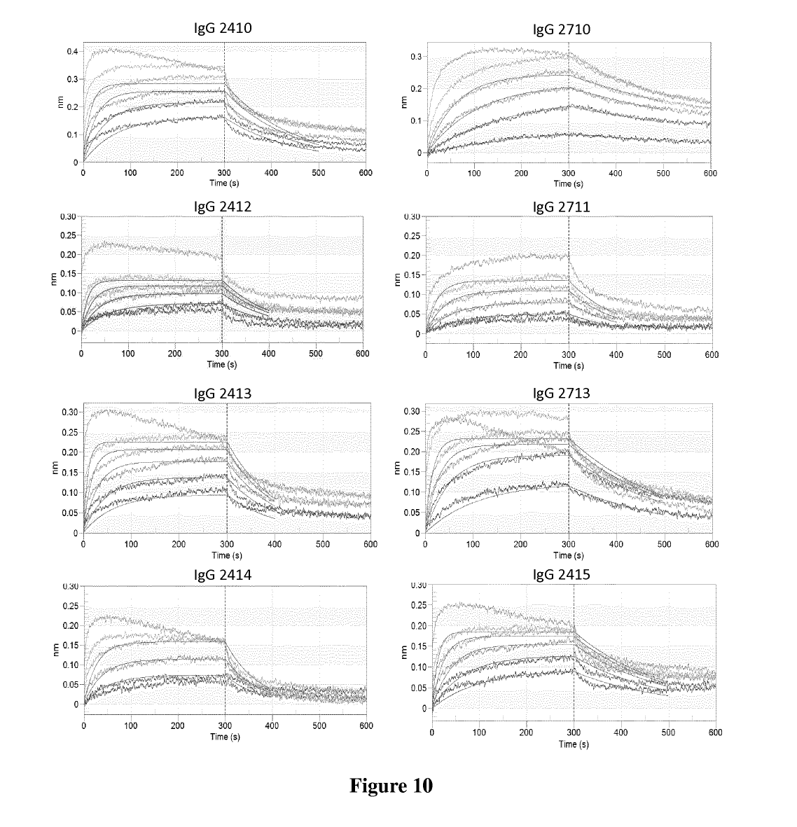

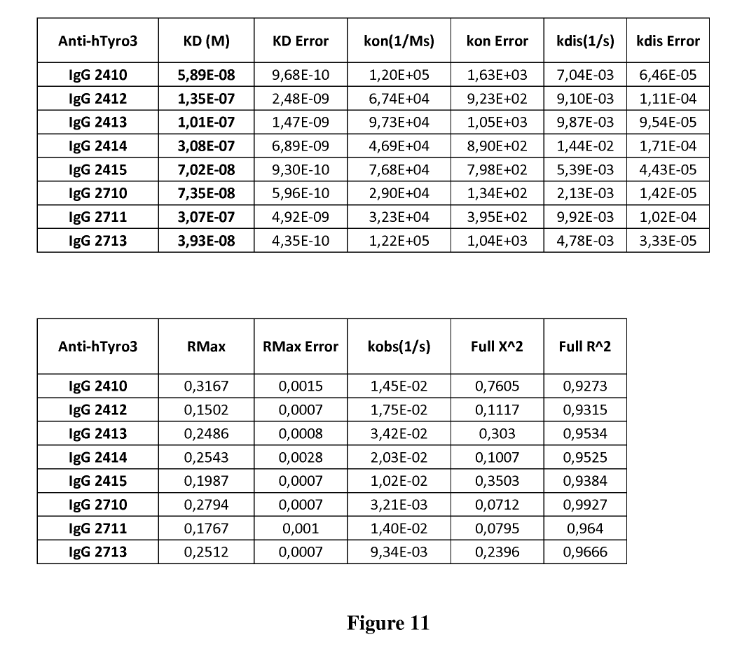

Affinity determinations were performed using biolayer interferometry on an Octet K2 instrument (Pall/ForteBio) equipped with anti-Human Kinetics (AHC) disposable fiber-optic biosensor tips. All experiments were conducted at 30° C. in the recommended Fortebio running buffer (PBS with 0.1% (w/v) bovine serum albumin (BSA) and 0.02% (v/v) Tween_20). This buffer was also used for dilution of ligand (here the antibody) and of the analyte (here the extra cellular domain of human Tyro3 protein). Samples in a 96-well microplates (No. Black polypropylene low binding plates, Greiner Bio-One, Frickenhausen, Germany) were agitated at 1000 rpm. Using an AHC biosensor (Pall Collaboration; ref:18-5060), tips were saturated with 5.625 μg/mL (37.5 nM) of the different anti-hTYRO3 for 10 min, which typically resulted in capture levels of 0.3 nm. Kinetic constants were determined for each of the purified antibodies (molecular mass 150 kDa) by adding the extra cellular domain of human Tyro3 diluted at 6 concentrations (37.5 or 62.5 (according to experiment), and 125, 250, 500, 1000 & 2500 nM). Between measurements, the anti-human Tyro3 biosensor surfaces were regenerated by exposing them to 3 cycles of 5 sec in 10 mM glycine, pH 1.5, followed by 5 sec in PBS. Association phase was measured for 300 seconds, dissociation phase depending on the interaction for 300 seconds depending on dissociation step. All measurements were corrected for baseline drift using the double reference substraction, that is to say by subtracting a control sensor without ligand exposed to each concentration of the extra cellular domain of human Tyro3 protein and a reference well with ligand submitted to running buffer only. Affinity, association (kon) and dissociation rate constants (koff) for each antibody were calculated applying a 1:1 interaction model fitting global, Rmax linked by sensor) on the ForteBio data analysis software 9.0. Curves that could not be reliably fitted with the software (mostly full R2<0.925), usually caused by heterogeneous binding, were excluded from further analysis. The results are presented in FIGS. 10 and 11.

### Example 6: Specificity ELISA

Extracellular domains of hTyro 3 (859-DK-100), hAxl (154-AL-100) or hMer (891-MR-100) from R&D Systems were coated onto 96-wells microtiter plates (1 μg/well) in PBS and incubated overnight at 4° C. Plates were washed three times with PBS and blocked in PBS/3% BSA for 1 hour at RT. Plates were washed as above and scFvs were added at 1 μg/mL (diluted in PBS) and incubated for 1 hour at RT. Plates were washed three times with PBST (PBS/0.01% Tween-20) and secondary antibody anti-FLAG-HRP was added (Abcam, ab49763, 1/5000 dilution in blocking buffer) for 1 hour at RT. Plates were washed as above with PBST and signal was developed with Ultra-TMB reagent (Thermo Scientific, 34028).

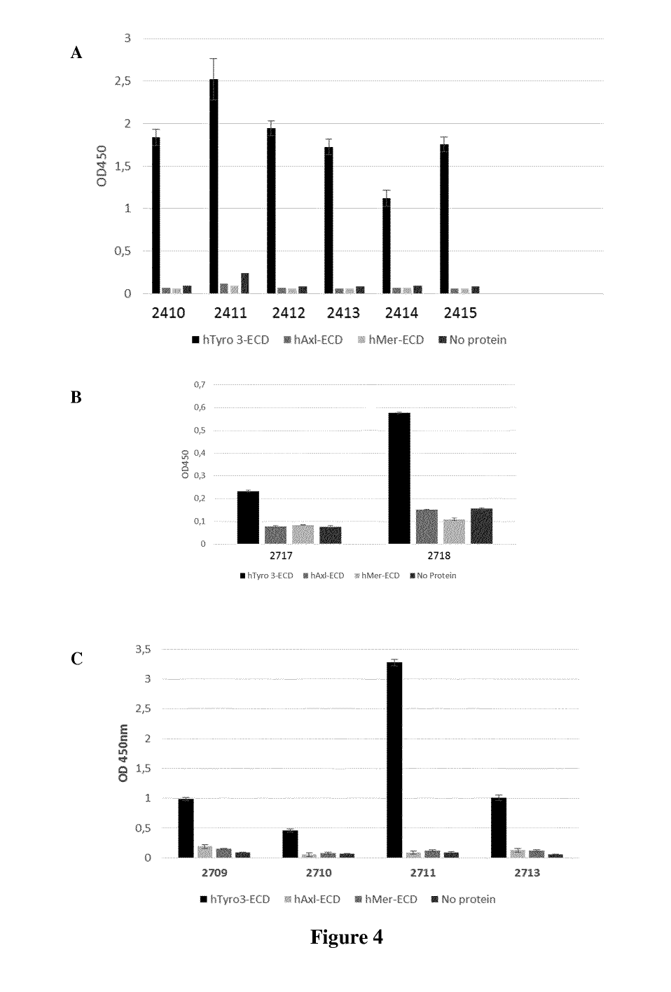

The results presented in FIG. 4 show that selected anti-Tyro3 scFvs do not cross-react with hAxl or hMer.

### Example 7: GAS6 Competition Assay

Microtiter plates were coated with human recombinant full-length Tyro3 protein at 2.5 μg/mL in PBS, 100 μl/well (OriGene # TP308260) and incubated overnight at 4° C. Plates were washed three times with PBS and blocked in PBS/3% BSA for 1 hour at RT. Plates were washed as above and antibodies (anti-Tyro3 scFvs or MAB859), biotinylated GAS6 or antibody/GAS6 mixtures were applied for 1 h at room temperature. Plates were washed three times with PBST (PBS/0.01% Tween-20) and secondary antibodies diluted in blocking buffer were added for 1 hour at RT. GAS6 binding to full-length Tyro3 was detected with 0.4 μg/mL of Streptavidin-HRP (Abcam, ab7403), ScFv binding with an anti-His-HRP antibody (Sigma-Aldrich, A7058, 1/5000 dilution) and MAB859 binding with anti-mouse-HRP (Abcam, ab6728 1/10000 dilution). Plates were rinsed with PBST and signal was developed with Ultra-TMB reagent (Thermo Scientific, 34028).

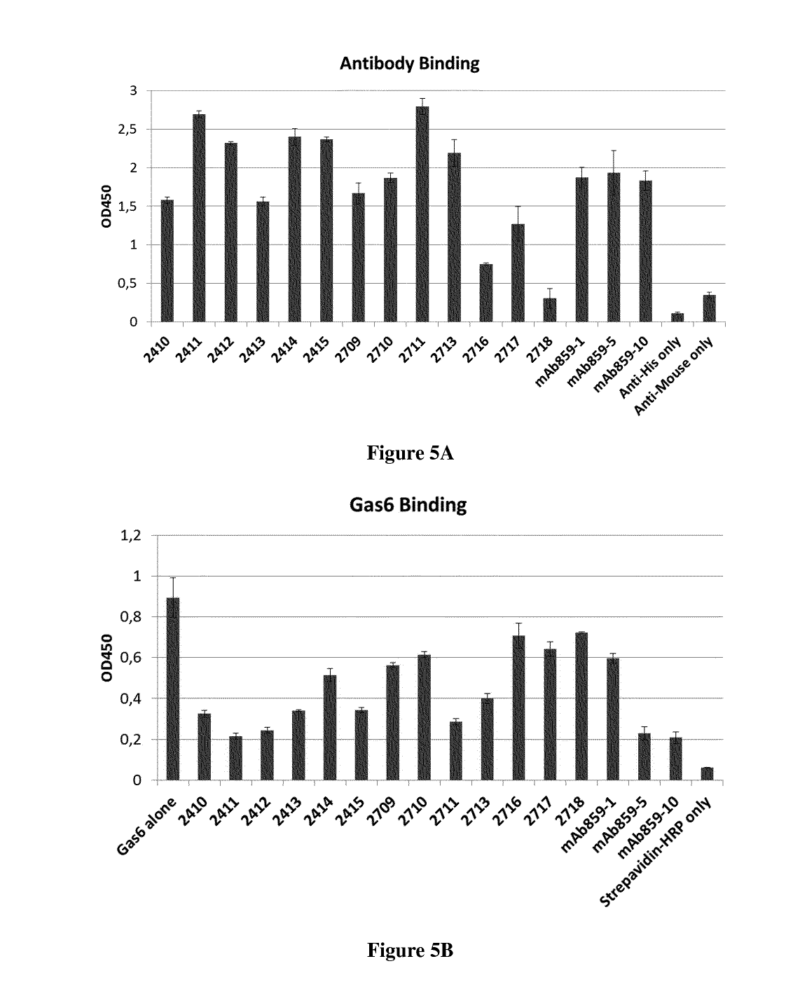

Results of these competition assays in the absence of GAS6 and in the presence of GAS6 are presented in FIGS. 5A and 5B, respectively.

### Example 8: Cross-Reactivity of Anti-Tyro3 scFvs and IgGs Against Cynomolgus and Murine Tyro3 ECD

The objective of this assay was to evaluate cross-reactivity of anti-Tyro3 scFvs and IgG against murine and cynomolgus Tyro3 proteins. This assay consists in measuring the binding of anti-Tyro3 antibodies on cynomolgus or murine Tyro3 ECD prepared as described in example 3, in a double sandwich ELISA as described in example 4 and illustrated in FIG. 2.

In these experiments, 100 μL of cynomolgus and murine Tyro3 strep tagged ECD proteins at the final dilution of 5 μg/mL (diluted in PBS, BSA 1% diluent solution) were added to each well (and so roughly 500 ng of Tyro3 proteins per well).

The primary and secondary antibodies and their used conditions are described in the table 16.

TABLE 16
Antibodies used to determine the cross-reactivity of anti-Tyro3 scFvs
and IgGs against cynomolgus and murine Tyro3 ECD

| Primary | | | |
|---|---|---|---|
| Antibody | Initial Dilution | Secondary antibody | Dilution |
| Anti-Tyro3 scFv | 10 μg/mL | | |
| Or irrelevant 735 | for human, | | |
| ScFv | cynomolgus and | | |
| | murine Tyro3 | | |
| Anti-Tyro3 scFv | | murine HRP conjugated | 1/10000 |
| Or irrelevant 735 | | anti Flag antibody (M2 | |
| ScFv | | from Sigma aldrich) | |
| Anti-Tyro3 IgG | 3 μg/mL for | | |
| or Control | human and | | |
| irrelevant | cynomolgus | | |
| IgG2656 | Tyro3, 10 μg/mL | | |
| | for murine Tyro3 | | |
| Anti-Tyro3 IgG | | goat HRP-coupled anti- | 1/75000 |
| or Control | | human IgG1 (kit from | |
| irrelevant | | Bethyl Laboratories) | |
| IgG2656 | | | |
| D38C6 Rabbit | 1/2000 | | |
| anti hTyro3 | | | |
| D38C6 Rabbit | | goat anti Rabbit HRP- | 1/10000 |
| anti hTyro3 | | coupled secondary | |
| | | antibody | |

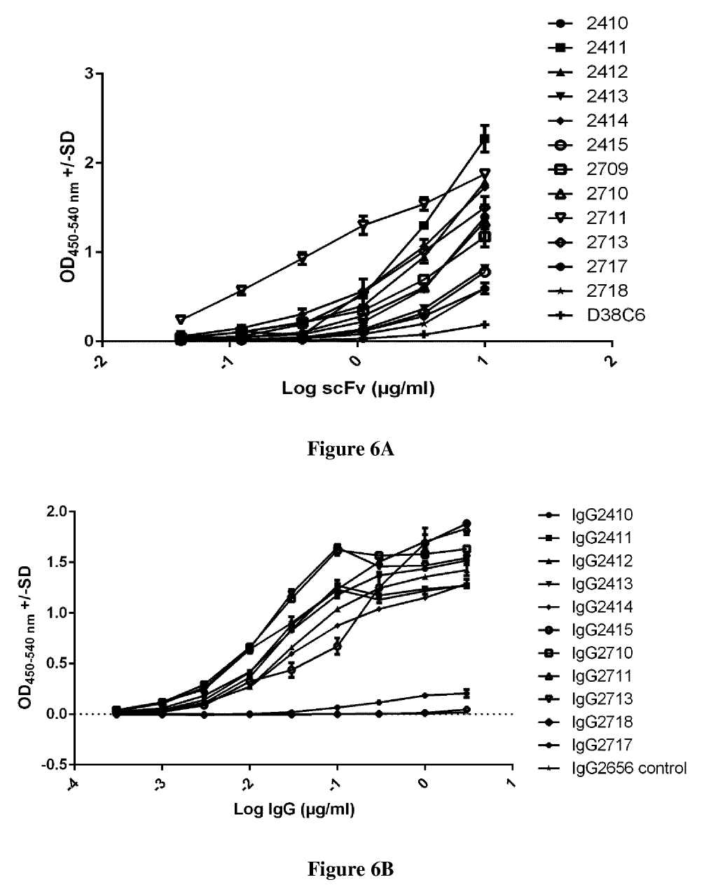

Cross-reactivity against cynomolgus Tyro3 ECD of anti-Tyro3 at the scFv format and at the IgG format are shown in FIGS. 6A and 6B, respectively.

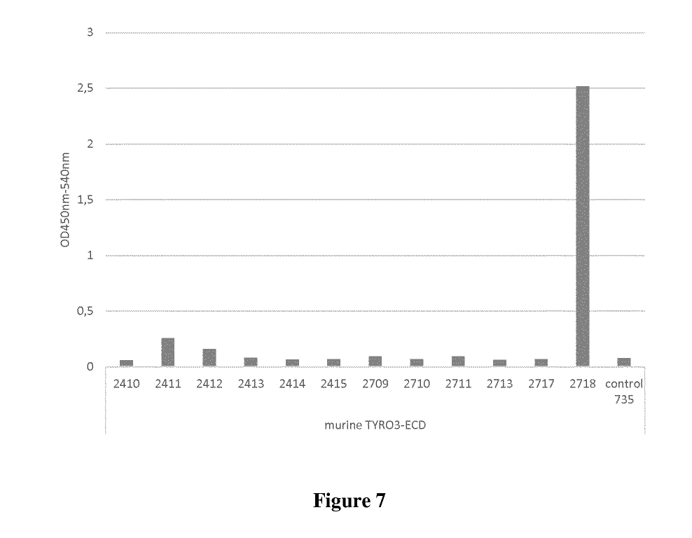

Cross-reactivity against murine Tyro3 ECD of anti-Tyro3 at the scFv format is shown in FIG. 7.

### Example 9 Epitope Mapping of Anti-Tyro3 scFvs

The objective of this assay was to define the binding region of each anti Tyro3 of the invention to a sub domain of the human Tyro3 ECD amongst the Ig like (Ig1 or Ig2) or fibronectin (FnIII 1 or 2) domains.

This assay consists in measuring the binding of anti-Tyro3 scFvs to different fragments of the human Tyro3 ECD. The double sandwich ELISA described in example 4 was used and roughly 500ng per well (100 μL/well at dilution of 5 μg/mL diluted in PBS, BSA 1%) of the produced Tyro3 fragments described in example 3, were immobilized.

The primary and secondary antibodies and their used conditions are described in the table 17.

TABLE 17
Antibodies used for the epitope mapping of Anti-Tyro3 scFvs.

| Anti-Tyro3 antibody | Initial Dilution | Secondary antibody | Dilution |
|---|---|---|---|
| Anti Tyro3 scFv | 10 μg/mL | | |
| Or irrelevant 735 ScFv | | | |
| Anti Tyro3 scFv | | M2-Anti DKK flag | 1/10000 |
| Or irrelevant 735 ScFv | | HRP | |

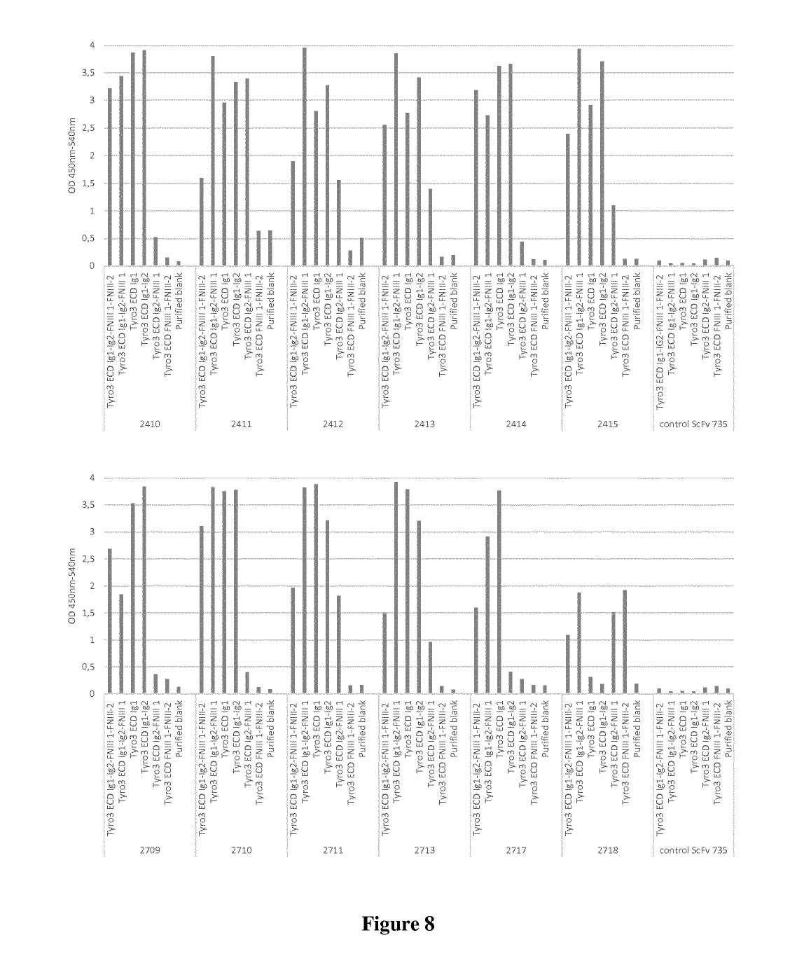

According to the binding results on the different domains of Tyro3 (as shown in FIG. 8), three sub-classes of Anti-Tyro3 antibodies have been identified:

the binders to Ig1 and to Ig2 (2411, 2412, 2413, 2415, 2711 and 2713);

the binders that bind Ig1 and that does not bind Ig2 (2410, 2414, 2709, 2710 and 2717) (the case of 2717 is particular, the epitope of 2717 is no more accessible in the Ig1-Ig2 fragment (binding to Ig1 and no binding at all for Ig1-Ig2 fragment) but this is no more the case when the others domains are present, for example in the full Tyro3 ECD);

and finally the binder to FNIII 1 domain (the 2718 ScFv).

### Example 10: Binding of Anti-Tyro 3 IgGs in Living Cells

To assess anti-Tyro3 IgG binding to cells overexpressing Tyro3, HEK293 cells transfected to express recombinant human Tyro3 and untransfected HEK293 cells expressing endogenous Tyro3 were used.

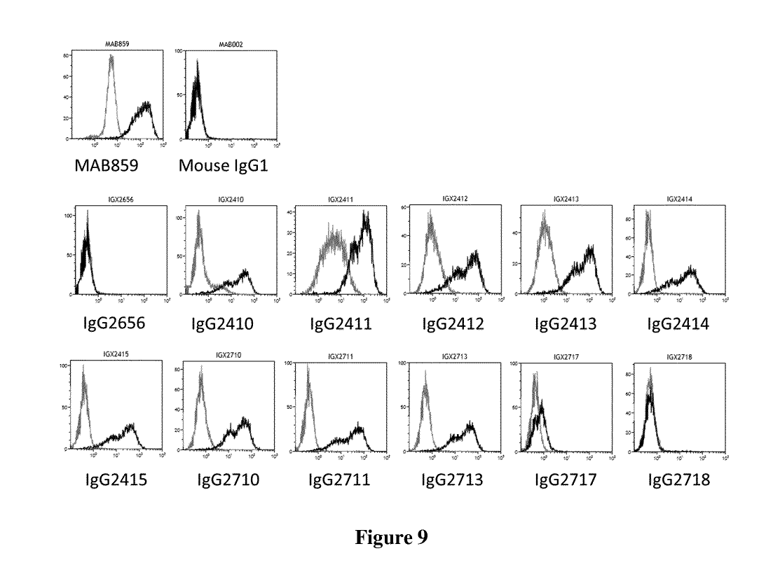

Tyro3 overexpression was controlled with exposition of 5^{E}5 cells to Live-Dead NIR dye (Life Technologies, #L10119) and 1 μg Tyro3-specific MAB859 monoclonal antibody (R&D Systems) or its isotype control (MAB002, R&D systems) in PBS for 30 minutes at 4° C. Cells were PBS-washed and incubated with 1:100 PE-conjugated Goat-anti Mouse Ig Ab (BD Pharmingen) in 100 μL PBS for 30 minutes at 4° C. Cells were washed and PE fluorescence of at least 10,000 live cells was immediately recorded on an ARIA III flow cytometer (Beckton Dickinson) (top panel of FIG. 9).

To determine the individual IgG ability to further bind Tyro3-transfected cells than untransfected cells, 5^{E}5 untransfected and Tyro3-transfected cells were exposed to Live-Dead NIR dye (Life Technologies, #L10119) and 1 μg of each IgX in PBS for 30 minutes at 4° C. Cells were PBS-washed and incubated with 20 μL PE-conjugated Mouse-anti Human Lambda Light Chain Ab (BD Pharmingen) in 100 μL PBS for 30 minutes at 4° C. Cells were washed and PE fluorescence of at least 10,000 live cells was immediately recorded on an ARIA III flow cytometer (Beckton Dickinson) (middle and bottom panels of FIG. 9). IgG ability to bind overexpressed Tyro3 was determined with overlay histograms showing PE expression profile of untransfected and Tyro3-transfected cell. The overlay histogram plots were obtained by using Kaluza software (Beckman Coulter) and is restricted to cells with the characteristic size and the granulometry of HEK cells, of which, dead cells were excluded (not shown).

1-15. (canceled)

16. An isolated human or humanized antibody that binds to the immunoglobulin-like domain Ig-1 of human Tyro3 receptor, does not bind to human Axl and Mer receptors, and reduces or inhibits the binding of human Gas6 to human Tyro3 receptor.

17. The antibody of claim 16, which antibody comprises a CDR-L1 consisting, or consisting essentially, of the CDR-L 1 of the light chain variable region of SEQ ID NO: 10 and/or comprises a CDR-H1 consisting, or consisting essentially, of the CDR-H1 of the heavy chain variable region of SEQ ID NO: 11.

18. The antibody of claim 16, which antibody further binds to the immunoglobulin-like domain Ig-2 of human Tyro3.

19. The antibody of claim 16, which antibody comprises
a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 88, 102, 116, 130, 144 or 158, and
a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 89, 103, 117, 131, 145 or 159.

20. The antibody of claim 16, which antibody does not bind to the immunoglobulin-like domain Ig-2 of humanTyro3.

21. The antibody of claim 16, which antibody comprises
a light chain comprising a CDR-L1 consisting, or consisting essentially, of the CDR-L1 of the light chain variable region of SEQ ID NO: 10, and a CDR-L2 and a CDR-L3 consisting, or consisting essentially, of the CDR-L2 and the CDR-L3 of the light chain variable region of SEQ ID NO: 10, 32, 46, 60 or 74, and
a heavy chain comprising a CDR-H1 consisting, or consisting essentially, of the CDR-H1 of the heavy chain variable region of SEQ ID NO: 11, and a CDR-H2 and a CDR-H3 consisting, or consisting essentially, of the CDR-H2 and the CDR-H3 of the heavy chain variable region of SEQ ID NO: 11, 33, 47, 61 or 75.

22. The antibody of claim 16, which antibody comprises
a light chain comprising CDR-L1, CDR-L2 and CDR-L3 consisting, or consisting essentially, of CDR-L1, CDR-L2 and CDR-L3 of the light chain variable region of SEQ ID NO: 10, and a heavy chain comprising CDR-H1, CDR-H2 and CDR-H3 consisting, or consisting essentially, of CDR-H1, CDR-H2 and CDR-H3 of the heavy chain variable region of SEQ ID NO: 11, or
a light chain comprising CDR-L1, CDR-L2 and CDR-L3 consisting, or consisting essentially, of CDR-L1, CDR-L2 and CDR-L3 of the light chain variable region of SEQ ID NO: 32, and a heavy chain comprising CDR-H1, CDR-H2 and CDR-H3 consisting, or consisting essentially, of CDR-H1, CDR-H2 and CDR-H3 of the heavy chain variable region of SEQ ID NO: 33, or
a light chain comprising CDR-L1, CDR-L2 and CDR-L3 consisting, or consisting essentially, of CDR-L1, CDR-L2 and CDR-L3 of the light chain variable region of SEQ ID NO: 46, and a heavy chain comprising CDR-H1, CDR-H2 and CDR-H3 consisting, or consisting essentially, of CDR-H1, CDR-H2 and CDR-H3 of the heavy chain variable region of SEQ ID NO: 47, or
a light chain comprising CDR-L1, CDR-L2 and CDR-L3 consisting, or consisting essentially, of CDR-L1, CDR-L2 and CDR-L3 of the light chain variable region of SEQ ID NO: 60, and a heavy chain comprising CDR-H1, CDR-H2 and CDR-H3 consisting, or consisting essentially, of CDR-H1, CDR-H2 and CDR-H3 of the heavy chain variable region of SEQ ID NO: 61, or
a light chain comprising CDR-L1, CDR-L2 and CDR-L3 consisting, or consisting essentially, of CDR-L1, CDR-L2 and CDR-L3 of the light chain variable region of SEQ ID NO: 74, and a heavy chain comprising CDR-H1, CDR-H2 and CDR-H3 consisting, or consisting essentially, of CDR-H1, CDR-H2 and CDR-H3 of the heavy chain variable region of SEQ ID NO: 75, or
a light chain comprising CDR-L1, CDR-L2 and CDR-L3 consisting, or consisting essentially, of CDR-L1, CDR-L2 and CDR-L3 of the light chain variable region of SEQ ID NO: 88, and a heavy chain comprising CDR-H1, CDR-H2 and CDR-H3 consisting, or consisting essentially, of CDR-H1, CDR-H2 and CDR-H3 of the heavy chain variable region of SEQ ID NO: 89, or
a light chain comprising CDR-L1, CDR-L2 and CDR-L3 consisting, or consisting essentially, of CDR-L1, CDR-L2 and CDR-L3 of the light chain variable region of SEQ ID NO: 102, and a heavy chain comprising CDR-H1, CDR-H2 and CDR-H3 consisting, or consisting essentially, of CDR-H1, CDR-H2 and CDR-H3 of the heavy chain variable region of SEQ ID NO: 103, or
a light chain comprising CDR-L1, CDR-L2 and CDR-L3 consisting, or consisting essentially, of CDR-L1, CDR-L2 and CDR-L3 of the light chain variable region of SEQ ID NO: 116, and a heavy chain comprising CDR-H1, CDR-H2 and CDR-H3 consisting, or consisting essentially, of CDR-H1, CDR-H2 and CDR-H3 of the heavy chain variable region of SEQ ID NO: 117, or
a light chain comprising CDR-L1, CDR-L2 and CDR-L3 consisting, or consisting essentially, of CDR-L1, CDR-L2 and CDR-L3 of the light chain variable region of SEQ ID NO: 130, and a heavy chain comprising CDR-H1, CDR-H2 and CDR-H3 consisting, or consisting essentially, of CDR-H1, CDR-H2 and CDR-H3 of the heavy chain variable region of SEQ ID NO: 131, or
a light chain comprising CDR-L1, CDR-L2 and CDR-L3 consisting, or consisting essentially, of CDR-L1, CDR-L2 and CDR-L3 of the light chain variable region of SEQ ID NO: 144, and a heavy chain comprising CDR-H1, CDR-H2 and CDR-H3 consisting, or consisting essentially, of CDR-H1, CDR-H2 and CDR-H3 of the heavy chain variable region of SEQ ID NO: 145, or
a light chain comprising CDR-L1, CDR-L2 and CDR-L3 consisting, or consisting essentially, of CDR-L1, CDR-L2 and CDR-L3 of the light chain variable region of SEQ ID NO: 158, and a heavy chain comprising CDR-H1, CDR-H2 and CDR-H3 consisting, or consisting essentially, of CDR-H1, CDR-H2 and CDR-H3 of the heavy chain variable region of SEQ ID NO: 159.

23. The antibody of claim 16, which antibody is an inhibitor of the Tyro3 receptor.

24. The antibody of claim 16, which antibody is an IgG1, IgG2, IgG3 or IgG4 antibody.

25. An isolated nucleic acid molecule selected from the group consisting of
(a) a nucleic acid sequence encoding an antibody of claim 16, and
(b) a nucleic acid complementary to the sequences in (a).

26. A vector comprising the nucleic acid of claim 25.

27. A host cell comprising a nucleic acid of claim 25 or a vector comprising said nucleic acid.

28. A method for producing an antibody comprising culturing the cell of claim 27 under conditions suitable for expression of the antibody, and optionally recovering said antibody.

29. A pharmaceutical composition comprising the antibody of claim 16, and a pharmaceutically acceptable carrier or excipient.

30. A method of treating a hyperproliferative disease comprising the administration of an antibody of claim 16 to a subject having a hyperproliferative disease.

31. A method of treating an infectious disease comprising the administration of an antibody of claim 16 to a subject having an infectious disease.


SEQUENCE LISTING (194 sequences)

SEQ ID NO: 1 - protein, 890 aa, Homo sapiens
  SIGNAL (1)..(40)
  MISC_FEATURE (41)..(128): immunoglobulin-like domain Ig-1
  MISC_FEATURE (139)..(220): immunoglobulin-like domain Ig-2
  MISC_FEATURE (227)..(320): Fibronectin type III-like domain FNIII-1
  MISC_FEATURE (325)..(416): Fibronectin type III-like domain FNIII-2
  MISC_FEATURE (518)..(790): Tyrosine kinase domain
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 2 - protein, 94 aa, Homo sapiens
AGLKLMGAPVKLTVSQGQPVKLNCSVEGMEEPDIQWVKDGAVVQNLDQLYIPVSEQHWIGFLSLKSVERSDAGRYWCQVEDGGETEISQPVWLT

SEQ ID NO: 3 - protein, 182 aa, Homo sapiens
AGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQ

SEQ ID NO: 4 - protein, 184 aa, Homo sapiens
VPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLA

SEQ ID NO: 5 - protein, 191 aa, Homo sapiens
AAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDR

SEQ ID NO: 6 - protein, 894 aa, Homo sapiens
  SIGNAL (1)..(25)
  DOMAIN (26)..(451): Extracellular domain
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 7 - protein, 999 aa, Homo sapiens
  SIGNAL (1)..(20)
  DOMAIN (21)..(505): Extracellular domain
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 8 - protein, 880 aa, Mus musculus
  SIGNAL (1)..(30)
  DOMAIN (31)..(419): Extracellular domain
MALRRSMGWPGLRPLLLAGLASLLLPGSAAAGLKLMGAPVKMTVSQGQPVKLNCSVEGMEDPDIHWMKDGTVVQNASQVSISISEHSWIGLLSLKSVERSDAGLYWCQVKDGEETKISQSVWLTVEGVPFFTVEPKDLAVPPNAPFQLSCEAVGPPEPVTIYWWRGLTKVGGPAPSPSVLNVTGVTQRTEFSCEARNIKGLATSRPAIVRLQAPPAAPFNTTVTTISSYNASVAWVPGADGLALLHSCTVQVAHAPGEWEALAVVVPVPPFTCLLRNLAPATNYSLRVRCANALGPSPYGDWVPFQTKGLAPARAPQNFHAIRTDSGLILEWEEVIPEDPGEGPLGPYKLSWVQENGTQDELMVEGTRANLTDWDPQKDLILRVCASNAIGDGPWSQPLVVSSHDHAGRQGPPHSRTSWVPVVLGVLTALITAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLVRFMVDIACGMEYLSSRNFIHRDLAARNCMLAEDMTVCVADFGLSRKIYSGDYYRQGCASKLPVKWLALESLADNLYTVHSDVWAFGVTMWEIMTRGQTPYAGIENAEIYNYLIGGNRLKQPPECMEEVYDLMYQCWSADPKQRPSFTCLRMELENILGHLSVLSTSQDPLYINIERAEQPTESGSPEVHCGERSSSEAGDGSGVGAVGGIPSDSRYIFSPGGLSESPGQLEQQPESPLNENQRLLLLQQGLLPHSSC

SEQ ID NO: 9 - protein, 409 aa, Macaca fascicularis
  SIGNAL (1)..(20)
  DOMAIN (21)..(409): extracellular domain
METDTLLLWVLLLWVPGSTGAGLKLMGAPVKLTVSQGQPVKLNCSVEGMEEPDIQWVKDGAVVQNLDQLYIPVSEQHWIGFLSLKSAERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNVTVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEGPLEGPLGPYKLSWVQDNGTQDELTVEGTRANLTGWDPQKDLIVRVCVSNAVGCGPWSQPLVVSSHDHAGQQGPPHSRTSW

SEQ ID NO: 10 - protein, 108 aa, Artificial Sequence
  Light chain variable region of IGX2410
SYELTQPPSVSVSPGQTARITCSGDALPKKYAYWYQQKSGQAPVLVIYEDEKRASGIPERFSGSSSGTMATLTISGAQVEDEADYYCYSQERNGNYAVFGGGTKLTVL

SEQ ID NO: 11 - protein, 125 aa, Artificial Sequence
  Heavy chain variable region of IGX2410
EVQLVQSGADVKKPGASLTISCQASGYTFTDYWISWVRQKPGKGLEWMGQIVPRASRTNYGPSFQGHVTISADRSTATAYLQWRSLEASDTAMYYCVRPYFQTSGGTIPEYYQHWGPGTLVTVSS

SEQ ID NO: 12 - protein, 11 aa, Artificial Sequence
  Sequence of CDR-L1 according to Chothia, AbM or Kabat definition
SGDALPKKYAY

SEQ ID NO: 13 - protein, 7 aa, Artificial Sequence
  Sequence of CDR-L1 according to Contact definition
KKYAYWY

SEQ ID NO: 14 - protein, 6 aa, Artificial Sequence
  Sequence of CDR-L1 according to IMGT definition
ALPKKY

SEQ ID NO: 15 - protein, 7 aa, Artificial Sequence
  Sequence of CDR-L2 of IGX2410 according to Chothia, AbM or Kabat definition
EDEKRAS

SEQ ID NO: 16 - protein, 10 aa, Artificial Sequence
  Sequence of CDR-L2 of IGX2410 according to Contact definition
LVIYEDEKRA

SEQ ID NO: 17 - protein, 11 aa, Artificial Sequence
  Sequence of CDR-L3 of IGX2410 according to Chothia, AbM, Kabat or IMGT definition
YSQERNGNYAV

SEQ ID NO: 18 - protein, 10 aa, Artificial Sequence
  Sequence of CDR-L3 of IGX2410 according to Contact definition
YSQERNGNYA

SEQ ID NO: 19 - protein, 7 aa, Artificial Sequence
  Sequence of CDR-H1 according to Chothia definition
GYTFTDY

SEQ ID NO: 20 - protein, 10 aa, Artificial Sequence
  Sequence of CDR-H1 according to AbM definition
GYTFTDYWIS

SEQ ID NO: 21 - protein, 5 aa, Artificial Sequence
  Sequence of CDR-H1 according to Kabat definition
DYWIS

SEQ ID NO: 22 - protein, 6 aa, Artificial Sequence
  Sequence of CDR-H1 according to Contact definition
TDYWIS

SEQ ID NO: 23 - protein, 8 aa, Artificial Sequence
  Sequence of CDR-H1 according to IMGT definition
GYTFTDYW

SEQ ID NO: 24 - protein, 6 aa, Artificial Sequence
  Sequence of CDR-H2 of IGX2410 according to Chothia definition
VPRASR

SEQ ID NO: 25 - protein, 10 aa, Artificial Sequence
  Sequence of CDR-H2 of IGX2410 according to AbM definition
QIVPRASRTN

SEQ ID NO: 26 - protein, 17 aa, Artificial Sequence
  Sequence of CDR-H2 of IGX2410 according to Kabat definition
QIVPRASRTNYGPSFQG

SEQ ID NO: 27 - protein, 13 aa, Artificial Sequence
  Sequence of CDR-H2 of IGX2410 according to Contact definition
WMGQIVPRASRTN

SEQ ID NO: 28 - protein, 8 aa, Artificial Sequence
  Sequence of CDR-H2 of IGX2410 according to IMGT definition
IVPRASRT

SEQ ID NO: 29 - protein, 16 aa, Artificial Sequence
  Sequence of CDR-H3 of IGX2410 according to Chothia, AbM or Kabat definition
PYFQTSGGTIPEYYQH

SEQ ID NO: 30 - protein, 17 aa, Artificial Sequence
  Sequence of CDR-H3 of IGX2410 according to Contact definition
VRPYFQTSGGTIPEYYQ

SEQ ID NO: 31 - protein, 18 aa, Artificial Sequence
  Sequence of CDR-H3 of IGX2410 according to IMGT definition
VRPYFQTSGGTIPEYYQH

SEQ ID NO: 32 - protein, 108 aa, Artificial Sequence
  Light chain variable region of IGX2414
SYELTQPPSVSVSPGQTARITCSGDALPKKYAYWYQQKSGQAPVLVIYEDWKRASGIPERFSGSSSGTMATLTISGAQVEDEADYYCYSQERKGNYFVFGGGTKLTVL

SEQ ID NO: 33 - protein, 125 aa, Artificial Sequence
  Heavy chain variable region of IGX2414
EVQLVQSGADVKKPGASLTISCQASGYTFTDYWISWVRQKPGKGLEWMGQINRHASNTMYGPSFQGHVTISADRSTATAYLQWRSLEASDTAMYYCVRSGGATSGGTIPEYYQHWGPGTLVTVSS

SEQ ID NO: 34 - protein, 7 aa, Artificial Sequence
  Sequence of CDR-L2 of IGX2414 according to Chothia, AbM or Kabat definition
EDWKRAS

SEQ ID NO: 35 - protein, 10 aa, Artificial Sequence
  Sequence of CDR-L2 of IGX2414 according to Contact definition
LVIYEDWKRA

SEQ ID NO: 36 - protein, 11 aa, Artificial Sequence
  Sequence of CDR-L3 of IGX2414 according to Chothia, AbM, Kabat or IMGT definition
YSQERKGNYFV

SEQ ID NO: 37 - protein, 10 aa, Artificial Sequence
  Sequence of CDR-L3 of IGX2414 according to Contact definition
YSQERKGNYF

SEQ ID NO: 38 - protein, 6 aa, Artificial Sequence
  Sequence of CDR-H2 of IGX2414 according to Chothia definition
NRHASN

SEQ ID NO: 39 - protein, 10 aa, Artificial Sequence
  Sequence of CDR-H2 of IGX2414 according to AbM definition
QINRHASNTM

SEQ ID NO: 40 - protein, 17 aa, Artificial Sequence
  Sequence of CDR-H2 of IGX2414 according to Kabat definition
QINRHASNTMYGPSFQG

SEQ ID NO: 41 - protein, 13 aa, Artificial Sequence
  Sequence of CDR-H2 of IGX2414 according to Contact definition
WMGQINRHASNTM

SEQ ID NO: 42 - protein, 8 aa, Artificial Sequence
  Sequence of CDR-H2 of IGX2414 according to IMGT definition
INRHASNT

SEQ ID NO: 43 - protein, 16 aa, Artificial Sequence
  Sequence of CDR-H3 of IGX2414 according to Chothia, AbM or Kabat definition
SGGATSGGTIPEYYQH

SEQ ID NO: 44 - protein, 17 aa, Artificial Sequence
  Sequence of CDR-H3 of IGX2414 according to Contact definition
VRSGGATSGGTIPEYYQ

SEQ ID NO: 45 - protein, 18 aa, Artificial Sequence
  Sequence of CDR-H3 of IGX2414 according to IMGT definition
VRSGGATSGGTIPEYYQH

SEQ ID NO: 46 - protein, 108 aa, Artificial Sequence
  Light chain variable region of IGX2709
SYELTQPPSVSVSPGQTARITCSGDALPKKYAYWYQQKSGQAPVLVIYEDKKRVSGIPERFSGSSSGTMATLTISGAQVEDEADYYCYSLAVDGNYGVFGGGTKLTVL

SEQ ID NO: 47 - protein, 127 aa, Artificial Sequence
  Heavy chain variable region of IGX2709
EVQLVQSGADVKKPGASLTISCQASGYTFTDYWISWVRQKPGKGLEWMGAISRNGSTTYYGPSFQGHVTISADRSTATAYLQWRSLEASDTAMYYCVRSGGLTSGGTIPEYYQHWGPGTLVPVSSVD

SEQ ID NO: 48 - protein, 7 aa, Artificial Sequence
  Sequence of CDR-L2 of IGX2709 according to Chothia, AbM or Kabat definition
EDKKRVS

SEQ ID NO: 49 - protein, 10 aa, Artificial Sequence
  Sequence of CDR-L2 of IGX2709 according to Contact definition
LVIYEDKKRV

SEQ ID NO: 50 - protein, 11 aa, Artificial Sequence
  Sequence of CDR-L3 of IGX2709 according to Chothia, AbM, Kabat or IMGT definition
YSLAVDGNYGV

SEQ ID NO: 51 - protein, 10 aa, Artificial Sequence
  Sequence of CDR-L3 of IGX2709 according to Contact definition
YSLAVDGNYG

SEQ ID NO: 52 - protein, 6 aa, Artificial Sequence
  Sequence of CDR-H2 of IGX2709 according to Chothia definition
SRNGST

SEQ ID NO: 53 - protein, 10 aa, Artificial Sequence
  Sequence of CDR-H2 of IGX2709 according to AbM definition
AISRNGSTTY

SEQ ID NO: 54 - protein, 17 aa, Artificial Sequence
  Sequence of CDR-H2 of IGX2709 according to Kabat definition
AISRNGSTTYYGPSFQG

SEQ ID NO: 55 - protein, 13 aa, Artificial Sequence
  Sequence of CDR-H2 of IGX2709 according to Contact definition
WMGAISRNGSTTY

SEQ ID NO: 56 - protein, 8 aa, Artificial Sequence
  Sequence of CDR-H2 of IGX2709 according to IMGT definition
ISRNGSTT

SEQ ID NO: 57 - protein, 16 aa, Artificial Sequence
  Sequence of CDR-H3 of IGX2709 according to Chothia, AbM or Kabat definition
SGGLTSGGTIPEYYQH

SEQ ID NO: 58 - protein, 17 aa, Artificial Sequence
  Sequence of CDR-H3 of IGX2709 according to Contact definition
VRSGGLTSGGTIPEYYQ

SEQ ID NO: 59 - protein, 18 aa, Artificial Sequence
  Sequence of CDR-H3 of IGX2709 according to IMGT definition
VRSGGLTSGGTIPEYYQH

SEQ ID NO: 60 - protein, 108 aa, Artificial Sequence
  Light chain variable region of IGX2710
SYELTQPPSVSVSPGQTARITCSGDALPKKYAYWYQQKSGQAPVLVIYEDSKRPSGIPERFSGSSSGTMATLTISGAQVEDEADYYCYSQNPQGNYFVFGGGTKLTVL

SEQ ID NO: 61 - protein, 125 aa, Artificial Sequence
  Heavy chain variable region of IGX2710
EVQLVQSGADVKKPGASLTISCQASGYTFTDYWISWVRQKPGKGLEWMGVINKYASWTRYGPSFQGHVTISADRSTATAYLQWRSLEASDTAMYYCVRSGGLTSGGTIPEYYQHWGPGTLVTVSS

SEQ ID NO: 62 - protein, 7 aa, Artificial Sequence
  Sequence of CDR-L2 of IGX2710 according to Chothia, AbM or Kabat definition
EDSKRPS

SEQ ID NO: 63 - protein, 10 aa, Artificial Sequence
  Sequence of CDR-L2 of IGX2710 according to Contact definition
LVIYEDSKRP

SEQ ID NO: 64 - protein, 11 aa, Artificial Sequence
  Sequence of CDR-L3 of IGX2710 according to Chothia, AbM, Kabat or IMGT definition
YSQNPQGNYFV

SEQ ID NO: 65 - protein, 10 aa, Artificial Sequence
  Sequence of CDR-L3 of IGX2710 according to Contact definition
YSQNPQGNYF

SEQ ID NO: 66 - protein, 6 aa, Artificial Sequence
  Sequence of CDR-H2 of IGX2710 according to Chothia definition
NKYASW

SEQ ID NO: 67 - protein, 10 aa, Artificial Sequence
  Sequence of CDR-H2 of IGX2710 according to AbM definition
VINKYASWTR

SEQ ID NO: 68 - protein, 17 aa, Artificial Sequence
  Sequence of CDR-H2 of IGX2710 according to Kabat definition
VINKYASWTRYGPSFQG

SEQ ID NO: 69 - protein, 13 aa, Artificial Sequence
  Sequence of CDR-H2 of IGX2710 according to Contact definition
WMGVINKYASWTR

SEQ ID NO: 70 - protein, 8 aa, Artificial Sequence
  Sequence of CDR-H2 of IGX2710 according to IMGT definition
INKYASWT

SEQ ID NO: 71 - protein, 16 aa, Artificial Sequence
  Sequence of CDR-H3 of IGX2710 according to Chothia, AbM or Kabat definition
SGGLTSGGTIPEYYQH

SEQ ID NO: 72 - protein, 17 aa, Artificial Sequence
  Sequence of CDR-H3 of IGX2710 according to Contact definition
VRSGGLTSGGTIPEYYQ

SEQ ID NO: 73 - protein, 18 aa, Artificial Sequence
  Sequence of CDR-H3 of IGX2710 according to IMGT definition
VRSGGLTSGGTIPEYYQH

SEQ ID NO: 74 - protein, 108 aa, Artificial Sequence
  Light chain variable region of IGX2717
SYELTQPPSVSVSPGQTARITCSGDALPKKYAYWYQQKSGQAPVLVIYEDNKRGSGIPERFSGSSSGTMATLTISGAQVEDEADYYCYSKSPEGNYMVFGGGTKLTVL

SEQ ID NO: 75 - protein, 125 aa, Artificial Sequence
  Heavy chain variable region of IGX2717
EVQLVQSGADVKKPGASLTISCQASGYTFTDYWISWVRQKPGKGLEWMGEIFLPPSTTVYGPSFQGHVTISADRSTATAYLQWRSLEASDTAMYYCVRQTRLTSGGTIPEYYQHWGPGTLVTVSS

SEQ ID NO: 76 - protein, 7 aa, Artificial Sequence
  Sequence of CDR-L2 of IGX2717 according to Chothia, AbM or Kabat definition
EDNKRGS

SEQ ID NO: 77 - protein, 10 aa, Artificial Sequence
  Sequence of CDR-L2 of IGX2717 according to Contact definition
LVIYEDNKRG

SEQ ID NO: 78 - protein, 11 aa, Artificial Sequence
  sequence of CDR-L3 of IGX2717 according to Chothia, AbM, Kabat or IMGT definition
YSKSPEGNYMV

SEQ ID NO: 79 - protein, 10 aa, Artificial Sequence
  Sequence of CDR-L3 of IGX2717 according to Contact definition
YSKSPEGNYM

SEQ ID NO: 80 - protein, 6 aa, Artificial Sequence
  sequence of CDR-H2 of IGX2717 according to Chothia definition
FLPPST

SEQ ID NO: 81 - protein, 10 aa, Artificial Sequence
  Sequence of CDR-H2 of IGX2717 according to AbM definition
EIFLPPSTTV

SEQ ID NO: 82 - protein, 17 aa, Artificial Sequence
  Sequence of CDR-H2 of IGX2717 according to Kabat definition
EIFLPPSTTVYGPSFQG

SEQ ID NO: 83 - protein, 13 aa, Artificial Sequence
  Sequence of CDR-H2 of IGX2717 according to Contact definition
WMGEIFLPPSTTV

SEQ ID NO: 84 - protein, 8 aa, Artificial Sequence
  Sequence of CDR-H2 of IGX2717 according to IMGT definition
IFLPPSTT

SEQ ID NO: 85 - protein, 16 aa, Artificial Sequence
  Sequence of CDR-H3 of IGX2717 according to Chothia, AbM or Kabat definition
QTRLTSGGTIPEYYQH

SEQ ID NO: 86 - protein, 17 aa, Artificial Sequence
  Sequence of CDR-H3 of IGX2717 according to Contact definition
VRQTRLTSGGTIPEYYQ

SEQ ID NO: 87 - protein, 18 aa, Artificial Sequence
  Sequence of CDR-H3 of IGX2717 according to IMGT definition
VRQTRLTSGGTIPEYYQH

SEQ ID NO: 88 - protein, 108 aa, Artificial Sequence
  Light chain variable region of IGX2411
SYELTQPPSVSVSPGQTARITCSGDALPKKYAYWYQQKSGQAPVLVIYEDSKRSSGIPERFSGSSSGTMATLTISGAQVEDEADYYCYSANQRGNYMVFGGGTKLTVL

SEQ ID NO: 89 - protein, 116 aa, Artificial Sequence
  Heavy chain variable region of IGX2411
EVQLVQSGADVKKPGASLTISCQASGYTFTDYWISWVRQKPGKGLEWMGSITRSSSRTHYGPSFQGHVTISADRSTATAYLQWRSLEASDTAMYYCVRGYTHLRTWGPGTLVTVSS

SEQ ID NO: 90 - protein, 7 aa, Artificial Sequence
  sequence of CDR-L2 of IGX2411 according to Chothia, AbM or Kabat definition
EDSKRSS

SEQ ID NO: 91 - protein, 10 aa, Artificial Sequence
  sequence of CDR-L2 of IGX2411 according to Contact definition
LVIYEDSKRS

SEQ ID NO: 92 - protein, 11 aa, Artificial Sequence
  sequence of CDR-L3 of IGX2411 acording to Chothia, AbM, Kabat or IMGT definition
YSANQRGNYMV

SEQ ID NO: 93 - protein, 10 aa, Artificial Sequence
  Sequence of CDR-L3 of IGX2411 according to Contact definition
YSANQRGNYM

SEQ ID NO: 94 - protein, 6 aa, Artificial Sequence
  sequence of CDR-H2 of IGX2411 according to Chothia definition
TRSSSR

SEQ ID NO: 95 - protein, 10 aa, Artificial Sequence
  sequence of CDR-H2 of IGX2411 according to AbM definition
SITRSSSRTH

SEQ ID NO: 96 - protein, 17 aa, Artificial Sequence
  Sequence of CDR-H2 of IGX2411 according to Kabat definition
SITRSSSRTHYGPSFQG

SEQ ID NO: 97 - protein, 13 aa, Artificial Sequence
  Sequence of CDR-H2 of IGX2411 according to Contact definition
WMGSITRSSSRTH

SEQ ID NO: 98 - protein, 8 aa, Artificial Sequence
  sequence of CDR-H2 of IGX2411 according to IMGT definition
ITRSSSRT

SEQ ID NO: 99 - protein, 7 aa, Artificial Sequence
  Sequence of CDR-H3 of IGX2411 according to Chothia, AbM or Kabat definition
GYTHLRT

SEQ ID NO: 100 - protein, 8 aa, Artificial Sequence
  sequence of CDR-H3 of IGX2411 according to Contact definition
VRGYTHLR

SEQ ID NO: 101 - protein, 9 aa, Artificial Sequence
  sequence of CDR-H3 of IGX2411 according to IMGT definition
VRGYTHLRT

SEQ ID NO: 102 - protein, 108 aa, Artificial Sequence
  Light chain variable region of IGX2412
SYELTQPPSVSVSPGQTARITCSGDALPKKYAYWYQQKSGQAPVLVIYEDAKRVSGIPERFSGSSSGTMATLTISGAQVEDEADYYCYSASANGNYRVFGGGTKLTVL

SEQ ID NO: 103 - protein, 125 aa, Artificial Sequence
  Heavy chain variable region of IGX2412
EVQLVQSGADVKKPGASLTISCQASGYTFTDYWISWVRQKPGKGLEWMGRIRPLLSHTMYGPSFQGHVTISADRSTATAYLQWRSLEASDTAMYYCVRSWLATSGGTIPEYYQHWGPGTLVTVSS

SEQ ID NO: 104 - protein, 7 aa, Artificial Sequence
  Sequence of CDR-L2 of IGX2412 according to Chothia, AbM or Kabat definition
EDAKRVS

SEQ ID NO: 105 - protein, 10 aa, Artificial Sequence
  sequence of CDR-L2 of IGX2412 according to Contact definition
LVIYEDAKRV

SEQ ID NO: 106 - protein, 11 aa, Artificial Sequence
  sequence of CDR-L3 of IGX2412 according to Chothia, AbM, Kabat or IMGT definition
YSASANGNYRV

SEQ ID NO: 107 - protein, 10 aa, Artificial Sequence
  sequence of CDR-L3 of IGX2412 according to Contact definition
YSASANGNYR

SEQ ID NO: 108 - protein, 6 aa, Artificial Sequence
  Sequence of CDR-H2 of IGX2412 according to Chothia definition
RPLLSH

SEQ ID NO: 109 - protein, 10 aa, Artificial Sequence
  sequence of CDR-H2 of IGX2412 according to AbM definition
RIRPLLSHTM

SEQ ID NO: 110 - protein, 17 aa, Artificial Sequence
  sequence of CDR-H2 of IGX2412 according to Kabat definition
RIRPLLSHTMYGPSFQG

SEQ ID NO: 111 - protein, 13 aa, Artificial Sequence
  sequence of CDR-H2 of IGX2412 according to Contact definition
WMGRIRPLLSHTM

SEQ ID NO: 112 - protein, 8 aa, Artificial Sequence
  sequence of CDR-H2 of IGX2412 according to IMGT definition
IRPLLSHT

SEQ ID NO: 113 - protein, 16 aa, Artificial Sequence
  sequence of CDR-H3 of IGX2412 according to Chothia, AbM or Kabat definition
SWLATSGGTIPEYYQH

SEQ ID NO: 114 - protein, 17 aa, Artificial Sequence
  sequence of CDR-H3 of IGX2412 according to Contact definition
VRSWLATSGGTIPEYYQ

SEQ ID NO: 115 - protein, 18 aa, Artificial Sequence
  sequence of CDR-H3 of IGX2412 according to IMGT definition
VRSWLATSGGTIPEYYQH

SEQ ID NO: 116 - protein, 108 aa, Artificial Sequence
  Light chain variable region of IGX2413
SYELTQPPSVSVSPGQTARITCSGDALPKKYAYWYQQKSGQAPVLVIYEDDKRLSGIPERFSGSSSGTMATLTISGAQVEDEADYYCYSTEINGNYAVFGGGTKLTVL

SEQ ID NO: 117 - protein, 121 aa, Artificial Sequence
  Heavy chain variable region of IGX2413
EVQLVQSGADVKKPGASLTISCQASGYTFTDYWISWVRQKPGKGLEWMGRIHTYQSSTRYGPSFQGHVTISADRSTATAYLQWRSLEASDTAMYYCVRHHSTTTANPYSSWGPGTLVTVSS

SEQ ID NO: 118 - protein, 7 aa, Artificial Sequence
  Sequence of CDR-L2 of IGX2413 according to Chothia, AbM or Kabat definition
EDDKRLS

SEQ ID NO: 119 - protein, 10 aa, Artificial Sequence
  Sequence of CDR-L2 of IGX2413 according to Contact definition
LVIYEDDKRL

SEQ ID NO: 120 - protein, 11 aa, Artificial Sequence
  Sequence of CDR-L3 of IGX2413 according to Chothia, AbM, Kabat or IMGT definition
YSTEINGNYAV

SEQ ID NO: 121 - protein, 10 aa, Artificial Sequence
  Sequence of CDR-L3 of IGX2413 according to Contact definition
YSTEINGNYA

SEQ ID NO: 122 - protein, 6 aa, Artificial Sequence
  Sequence of CDR-H2 of IGX2413 according to Chothia definition
HTYQSS

SEQ ID NO: 123 - protein, 10 aa, Artificial Sequence
  Sequence of CDR-H2 of IGX2413 according to AbM definition
RIHTYQSSTR

SEQ ID NO: 124 - protein, 17 aa, Artificial Sequence
  Sequence of CDR-H2 of IGX2413 according to Kabat definition
RIHTYQSSTRYGPSFQG

SEQ ID NO: 125 - protein, 13 aa, Artificial Sequence
  Sequence of CDR-H2 of IGX2413 according to Contact definition
WMGRIHTYQSSTR

SEQ ID NO: 126 - protein, 8 aa, Artificial Sequence
  Sequence of CDR-H2 of IGX2413 according to IMGT definition
IHTYQSST

SEQ ID NO: 127 - protein, 12 aa, Artificial Sequence
  Sequence of CDR-H3 of IGX2413 according to Chothia, AbM or Kabat definition
HHSTTTANPYSS

SEQ ID NO: 128 - protein, 13 aa, Artificial Sequence
  Sequence of CDR-H3 of IGX2413 according to Contact definition
VRHHSTTTANPYS

SEQ ID NO: 129 - protein, 14 aa, Artificial Sequence
  Sequence of CDR-H3 of IGX2413 according to IMGT definition
VRHHSTTTANPYSS

SEQ ID NO: 130 - protein, 108 aa, Artificial Sequence
  Light chain variable region of IGX2415
SYELTQPPSVSVSPGQTARITCSGDALPKKYAYWYQQKSGQAPVLVIYEDKKRISGIPERFSGSSSGTMATLTISGAQVEDEADYYCYSTSTGGNYMVFGGGTKLTVL

SEQ ID NO: 131 - protein, 125 aa, Artificial Sequence
  Heavy chain variable region of IGX2415
EVQLVQSGADVKKPGASLTISCQASGYTFTDYWISWVRQKPGKGLEWMGQIRGPASATHYGPSFQGHVTISADRSTATAYLQWRSLEASDTAMYYCVRHQQQTSGGTIPEYYQHWGPGTLVTVSS

SEQ ID NO: 132 - protein, 7 aa, Artificial Sequence
  Sequence of CDR-L2 of IGX2415 according to Chothia, AbM or Kabat definition
EDKKRIS

SEQ ID NO: 133 - protein, 10 aa, Artificial Sequence
  Sequence of CDR-L2 of IGX2415 according to Contact definition
LVIYEDKKRI

SEQ ID NO: 134 - protein, 11 aa, Artificial Sequence
  Sequence of CDR-L3 of IGX2415 according to Chothia, AbM, Kabat or IMGT definition
YSTSTGGNYMV

SEQ ID NO: 135 - protein, 10 aa, Artificial Sequence
  Sequence of CDR-L3 of IGX2415 according to Contact definition
YSTSTGGNYM

SEQ ID NO: 136 - protein, 6 aa, Artificial Sequence
  Sequence of CDR-H2 of IGX2415 according to Chothia definition
RGPASA

SEQ ID NO: 137 - protein, 10 aa, Artificial Sequence
  Sequence of CDR-H2 of IGX2415 according to AbM definition
QIRGPASATH

SEQ ID NO: 138 - protein, 17 aa, Artificial Sequence
  Sequence of CDR-H2 of IGX2415 according to Kabat definition
QIRGPASATHYGPSFQG

SEQ ID NO: 139 - protein, 13 aa, Artificial Sequence
  Sequence of CDR-H2 of IGX2415 according to Contact definition
WMGQIRGPASATH

SEQ ID NO: 140 - protein, 8 aa, Artificial Sequence
  Sequence of CDR-H2 of IGX2415 according to IMGT definition
IRGPASAT

SEQ ID NO: 141 - protein, 16 aa, Artificial Sequence
  Sequence of CDR-H3 of IGX2415 according to Chothia, AbM or Kabat definition
HQQQTSGGTIPEYYQH

SEQ ID NO: 142 - protein, 17 aa, Artificial Sequence
  Sequence of CDR-H3 of IGX2415 according to Contact definition
VRHQQQTSGGTIPEYYQ

SEQ ID NO: 143 - protein, 18 aa, Artificial Sequence
  Sequence of CDR-H3 of IGX2415 according to IMGT definition
VRHQQQTSGGTIPEYYQH

SEQ ID NO: 144 - protein, 108 aa, Artificial Sequence
  Light chain variable region of IGX2711
SYELTQPPSVSVSPGQTARITCSGDALPKKYAYWYQQKSGQAPVLVIYEDAKRSSGIPERFSGSSSGTMATLTISGAQVEDEADYYCYSLSDSGNYFVFGGGTKLTVL

SEQ ID NO: 145 - protein, 125 aa, Artificial Sequence
  Heavy chain variable region of IGX2711
EVQLVQSGADVKKPGASLTISCQASGYTFTDYWISWVRQKPGKGLEWMGQIRGQHSSTLYGPSFQGHVTISADRSTATAYLQWRSLEASDTAMYYCVRHGASTSGGTIPEYYQHWGPGTLVTVSS

SEQ ID NO: 146 - protein, 7 aa, Artificial Sequence
  Sequence of CDR-L2 of IGX2711 according to Chothia, AbM or Kabat definition
EDAKRSS

SEQ ID NO: 147 - protein, 10 aa, Artificial Sequence
  Sequence of CDR-L2 of IGX2711 according to Contact definition
LVIYEDAKRS

SEQ ID NO: 148 - protein, 11 aa, Artificial Sequence
  Sequence of CDR-L3 of IGX2711 according to Chothia, AbM, Kabat or IMGT definition
YSLSDSGNYFV

SEQ ID NO: 149 - protein, 10 aa, Artificial Sequence
  Sequence of CDR-L3 of IGX2711 according to Contact definition
YSLSDSGNYF

SEQ ID NO: 150 - protein, 6 aa, Artificial Sequence
  Sequence of CDR-H2 of IGX2711 according to Chothia definition
RGQHSS

SEQ ID NO: 151 - protein, 10 aa, Artificial Sequence
  Sequence of CDR-H2 of IGX2711 according to AbM definition
QIRGQHSSTL

SEQ ID NO: 152 - protein, 17 aa, Artificial Sequence
  Sequence of CDR-H2 of IGX2711 according to Kabat definition
QIRGQHSSTLYGPSFQG

SEQ ID NO: 153 - protein, 13 aa, Artificial Sequence
  Sequence of CDR-H2 of IGX2711 according to Contact definition
WMGQIRGQHSSTL

SEQ ID NO: 154 - protein, 8 aa, Artificial Sequence
  Sequence of CDR-H2 of IGX2711 according to IMGT definition
IRGQHSST

SEQ ID NO: 155 - protein, 16 aa, Artificial Sequence
  Sequence of CDR-H3 of IGX2711 according to Chothia, AbM or Kabat definition
HGASTSGGTIPEYYQH

SEQ ID NO: 156 - protein, 17 aa, Artificial Sequence
  Sequence of CDR-H3 of IGX2711 according to Contact definition
VRHGASTSGGTIPEYYQ

SEQ ID NO: 157 - protein, 18 aa, Artificial Sequence
  Sequence of CDR-H3 of IGX2711 according to IMGT definition
VRHGASTSGGTIPEYYQH

SEQ ID NO: 158 - protein, 108 aa, Artificial Sequence
  Light chain variable region of IGX2713
SYELTQPPSVSVSPGQTARITCSGDALPKKYAYWYQQKSGQAPVLVIYEDHKRASGIPERFSGSSSGTMATLTISGAQVEDEADYYCYSISPAGNYGVFGGGTKLTVL

SEQ ID NO: 159 - protein, 125 aa, Artificial Sequence
  Heavy chain variable region of IGX2713
EVQLVQSGADVKKPGASLTISCQASGYTFTDYWISWVRQKPGKGLEWMGRISRHGSQTYYGPSFQGHVTISADRSTATAYLQWRSLEASDTAMYYCVRAGGQTSGGTIPEYYQHWGPGTLVTVSS

SEQ ID NO: 160 - protein, 7 aa, Artificial Sequence
  Sequence of CDR-L2 of IGX2713 according to Chothia, AbM or Kabat definition
EDHKRAS

SEQ ID NO: 161 - protein, 10 aa, Artificial Sequence
  Sequence of CDR-L2 of IGX2713 according to Contact definition
LVIYEDHKRA

SEQ ID NO: 162 - protein, 11 aa, Artificial Sequence
  Sequence of CDR-L3 of IGX2713 according to Chothia, AbM, Kabat or IMGT definition
YSISPAGNYGV

SEQ ID NO: 163 - protein, 10 aa, Artificial Sequence
  Sequence of CDR-L3 of IGX2713 according to Contact definition
YSISPAGNYG

SEQ ID NO: 164 - protein, 6 aa, Artificial Sequence
  Sequence of CDR-H2 of IGX2713 according to Chothia definition
SRHGSQ

SEQ ID NO: 165 - protein, 10 aa, Artificial Sequence
  Sequence of CDR-H2 of IGX2713 according to AbM definition
RISRHGSQTY

SEQ ID NO: 166 - protein, 17 aa, Artificial Sequence
  Sequence of CDR-H2 of IGX2713 according to Kabat definition
RISRHGSQTYYGPSFQG

SEQ ID NO: 167 - protein, 13 aa, Artificial Sequence
  Sequence of CDR-H2 of IGX2713 according to Contact definition
WMGRISRHGSQTY

SEQ ID NO: 168 - protein, 8 aa, Artificial Sequence
  Sequence of CDR-H2 of IGX2713 according to IMGT definition
ISRHGSQT

SEQ ID NO: 169 - protein, 16 aa, Artificial Sequence
  Sequence of CDR-H3 of IGX2713 according to Chothia, AbM or Kabat definition
AGGQTSGGTIPEYYQH

SEQ ID NO: 170 - protein, 17 aa, Artificial Sequence
  Sequence of CDR-H3 of IGX2713 according to Contact definition
VRAGGQTSGGTIPEYYQ

SEQ ID NO: 171 - protein, 18 aa, Artificial Sequence
  Sequence of CDR-H3 of IGX2713 according to IMGT definition
VRAGGQTSGGTIPEYYQH

SEQ ID NO: 172 - protein, 108 aa, Artificial Sequence
  Light chain variable region of IGX2718
SYELTQPPSVSVSPGQTARITCSGDALPKKYAYWYQQKSGQAPVLVIYEDTKRHSGIPERFSGSSSGTMATLTISGAQVEDEADYYCYSAEHRGNYAVFGGGTKLTVL

SEQ ID NO: 173 - protein, 121 aa, Artificial Sequence
  Heavy chain variable region of IGX2718
EVQLVQSGADVKKPGASLTISCQASGYTFTDYWISWVRQKPGKGLEWMGHINPMRSTTTYGPSFQGHVTISADRSTATAYLQWHSLEASDTAMYYCVRNDNHDDYTYTDDWGPGTLVTVSS

SEQ ID NO: 174 - protein, 7 aa, Artificial Sequence
  Sequence of CDR-L2 of IGX2718 according to Chothia, AbM or Kabat definition
EDTKRHS

SEQ ID NO: 175 - protein, 10 aa, Artificial Sequence
  Sequence of CDR-L2 of IGX2718 according to Contact definition
LVIYEDTKRH

SEQ ID NO: 176 - protein, 11 aa, Artificial Sequence
  Sequence of CDR-L3 of IGX2718 according to Chothia, AbM, Kabat or IMGT definition
YSAEHRGNYAV

SEQ ID NO: 177 - protein, 10 aa, Artificial Sequence
  Sequence of CDR-L3 of IGX2718 according to Contact definition
YSAEHRGNYA

SEQ ID NO: 178 - protein, 6 aa, Artificial Sequence
  Sequence of CDR-H2 of IGX2718 according to Chothia definition
NPMRST

SEQ ID NO: 179 - protein, 10 aa, Artificial Sequence
  Sequence of CDR-H2 of IGX2718 according to AbM definition
HINPMRSTTT

SEQ ID NO: 180 - protein, 17 aa, Artificial Sequence
  Sequence of CDR-H2 of IGX2718 according to Kabat definition
HINPMRSTTTYGPSFQG

SEQ ID NO: 181 - protein, 13 aa, Artificial Sequence
  Sequence of CDR-H2 of IGX2718 according to Contact definition
WMGHINPMRSTTT

SEQ ID NO: 182 - protein, 8 aa, Artificial Sequence
  Sequence of CDR-H2 of IGX2718 according to IMGT definition
INPMRSTT

SEQ ID NO: 183 - protein, 12 aa, Artificial Sequence
  Sequence of CDR-H3 of IGX2718 according to Chothia, AbM or Kabat definition
NDNHDDYTYTDD

SEQ ID NO: 184 - protein, 13 aa, Artificial Sequence
  Sequence of CDR-H3 of IGX2718 according to Contact definition
VRNDNHDDYTYTD

SEQ ID NO: 185 - protein, 14 aa, Artificial Sequence
  Sequence of CDR-H3 of IGX2718 according to IMGT definition
VRNDNHDDYTYTDD

SEQ ID NO: 186 - protein, 5 aa, Artificial Sequence
  Consensus sequence of CDR-L2
  MISC_FEATURE (3)..(3): X is E, W, K, S, N, A, D, H or T
  MISC_FEATURE (5)..(5): X is A, V, P, G, S, V, L, I or H
EDXRX

SEQ ID NO: 187 - protein, 11 aa, Artificial Sequence
  Consensus sequence of CDR-L3
  MISC_FEATURE (3)..(3): X is Q, L, K, A, T, L or I
  MISC_FEATURE (4)..(4): X is E, A, N or S
  MISC_FEATURE (5)..(5): X is R, V, P, Q, A, I, T, D or H
  MISC_FEATURE (6)..(6): X is N, K, D, Q, E, R, G, S or A
  MISC_FEATURE (10)..(10): X is A, F, G, M or R
YSXXXXGNYXV

SEQ ID NO: 188 - protein, 17 aa, Artificial Sequence
  Consensus sequence of CDR-H2
  MISC_FEATURE (1)..(1): X is Q, A, V, E, S, R or H
  MISC_FEATURE (3)..(3): X is V, N, S, F, T, R or H
  MISC_FEATURE (4)..(4): X is P, R, K, L, T or G
  MISC_FEATURE (5)..(5): X is R, H, N, Y, P, S, L, Q or M
  MISC_FEATURE (6)..(6): X is A, G, P, S, L, Q H or R
  MISC_FEATURE (8)..(8): X is R, N, T, W, H, S, A or Q
  MISC_FEATURE (10)..(10): X is N, M, Y, R, V, H, L or T
XIXXXXSXTXYGPSFQG

SEQ ID NO: 189 - protein, 16 aa, Artificial Sequence
  Consensus sequence of CDR-H3
  MISC_FEATURE (1)..(1): X is P, S, Q, H or A
  MISC_FEATURE (2)..(2): X is Y, G, T, W or Q
  MISC_FEATURE (3)..(3): X is F, G, R, L, Q or A
  MISC_FEATURE (4)..(4): X is Q, A, L or S
XXXXTSGGTIPEYYQH

SEQ ID NO: 190 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 192 - protein, 30 aa, Homo sapiens
DWVPFQTKGLAPASAPQNLHAIRTDSGLIL

SEQ ID NO: 193 - protein, 20 aa, Artificial Sequence
  signal peptide
METDTLLLWVLLLWVPGSTG

SEQ ID NO: 194 - protein, 26 aa, Artificial Sequence
  C-terminal Tag comprising a thrombine cleavage site followed by an octa-histidine (8xHis)-tag coupled to a streptavidin tag
LVPRGSSAHHHHHHHHSAWSHPQFEK